SEQUENCE LISTING (3 sequences)

SEQ ID NO: 1 - protein, 839 aa, Homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 2 - protein, 160 aa, Homo sapiens
MLPFLFFSTLFSSIFTEAQKQYWVCNSSDASISYTYCDKMQYPISINVNPCIELKGSKGLLHIFYIPRRDLKQLYFNLYITVNTMNLPKRKEVICRGSDDDYSFCRALKGETVNTTISFSFKGIKFSKGKYKCVVEAISGSPEEMLFCLEFVILHQPNSN

SEQ ID NO: 3 - protein, 5 aa, Artificial Sequence
  Linker sequence
GAAGG

(19) United States
(12) Patent Application Publication
(10) Pub. No.: US 2006/0241040 A1
(43) Pub. Date: Oct. 26, 2006

(54) Methods of treating disorders associated with toll-like receptor 4 (TLR4) signalling

(76) Inventors: Alberto Visintin, Worcester, MA (US); Douglas T. Golenbock, Wellesley, MA (US)

Correspondence Address: FISH & RICHARDSON PC, P.O. BOX 1022, MINNEAPOLIS, MN, 55440-1022, US

(21) Appl. No.: 11/399,274

(22) Filed: Apr. 6, 2006

Related U.S. Application Data
(60) Provisional application No. 60/668,703, filed on Apr. 6, 2005.

(51) Int. Cl. A61K 38/17

(52) U.S. Cl. 514/12

(57) ABSTRACT

Described herein are methods and compositions for treating, preventing, and diagnosing disorders associated with TLR4 signalling, e.g., gram negative bacterial infection and sterile inflammations such as rheumatoid arthritis.

### CLAIM OF PRIORITY

This application claims the benefit under 35 USC § 19(e) of U.S. Provisional Patent Application Ser. No. 60/668,703, filed on Apr. 6, 2005, the entire contents of which are hereby incorporated by reference.

### FEDERALLY SPONSORED RESEARCH OR DEVELOPMENT

This invention was made with Government support under Grant No. RO1 GM54060, RR14466 and AI52455 awarded by the National Institutes of Health, and DARPA grant ONR/N 00173-04-1-G018 awarded by the Department of Defense. The Government has certain rights in the invention.

### TECHNICAL FIELD

This invention relates to methods of treating disorders associated with Toll-Like Receptor (TLR) 4 signalling, e.g., sepsis and septic shock associated with gram-negative infections, as well as sterile inflammation, e.g., rheumatoid arthritis.

### BACKGROUND

The molecular “antenna” that recognizes and alerts mammalian cells to the presence of lipopolysaccharide (LPS), a bacterial endotoxin associated with sepsis and septic shock, is a receptor complex composed of Toll-Like Receptor 4 (TLR4) and Myeloid Differentiation Antigen-2 (MD-2). TLR4 is a type I transmembrane glycoprotein characterized by the presence of 22 leucine rich repeats (LRR) on the extracellular domain (1). Initiation of the signal elicited by LPS depends on the dimerization of the cytoplasmic TIR (Toll-Interleukin-1 Resistance) domain of TLR4 (2-3). The activation signal is then propagated by the recruitment of a dedicated array of intracellular signaling protein adaptors followed by the activation of a complex serine/threonine kinase cascade, which eventually leads to the transcription of immunologically relevant genes (5). Recognition and signaling of LPS strictly depends on MD-2 (6-8), a 160 amino acid secreted glycoprotein that co-precipitates with TLR4 (6). Viriyakosol and collaborators reported that soluble MD-2 binds LPS with an apparent Kd of 65 nM (9). Physical contact between TLR4 and LPS is still an unresolved and contentious issue. TLR4 can be captured by a biotinylated form of LPS only when MD-2 is provided as a soluble molecule, or when co-transfected with TLR4 (4). These results suggest that the minimal cell surface LPS signaling receptor complex consists of MD-2 and TLR4 (4, 10, 11). Supporting the idea that LPS and MD-2/TLR4 form a stable complex on the cell surface, antibodies exist that can recognize the MD-2/TLR4 complex in the LPS loaded or unloaded state (12).

MD-2 is an Ig domain folded protein belonging to the ML (MD-2-related lipid recognition) family of lipid binding receptors (13). Computational modeling suggests that MD-2 is capable of forming a barrel-like structure with a hydrophobic cavity large enough to accommodate the fatty acid moieties of lipid A (14, 15). A highly positively charged region of MD-2 that flanks this hypothetical hydrophobic cavity is required for stable binding to LPS. Mutations in the lysine residues of this region correlate with the loss of LPS binding and as a result, the loss of activity (4). Additional structural details necessary for MD-2 function have also been defined. For example, Cys95 is a critical residue for MD-2 activity (8). Cys95 is predicted to be located on the surface of the hypothetical barrel, as are all of the other six Cys residues save one, consistent with the idea that MD-2 is capable of forming covalently bound oligomers (4, 16, 17), while not precluding the existence of a monomeric form. Monomeric MD-2 has been reported to preferentially bind to a soluble TLR4 ectodomain (18).

### SUMMARY

The invention is based, at least in part, on the discovery that LPS-inhibitory lipid A analogs, such as the synthetic compound E5564, function by preventing LPS/MD-2 interactions. Thus, the invention includes methods for identifying improved LPS inhibitors, by identifying compounds that prevent binding of LPS to MD-2; in some embodiments, the compounds are analogs of lipid A or a portion thereof. Further, it was found that normal “healthy” human serum contains about 1.5 nM of functional soluble MD-2 (sMD-2), thus, the invention includes methods of diagnosing gram-negative bacterial infections by detecting elevated levels of sMD-2. Finally, it was discovered that a fusion protein including the extracellular portion of TLR4 linked to an Fc fragment (TLR4:Fc) is capable of blocking LPS-induced signalling in human peripheral monocytes. Thus, the invention also includes methods of treating disorders associated with gram-negative bacterial infections by administering a therapeutically effective amount of a composition including TLR4:Fc, and methods of identifying compounds that interfere with the TLR4/MD-2 interaction. The results described herein indicate that blocking MD-2, e.g., by chemical LPS antagonists or soluble decoy receptors (T4:Fc), inhibits TLR4 signaling. The methods generally include targeting MD-2, rather than TLR4.

In one aspect, the invention includes methods for treating or preventing a disorder associated with a gram negative bacterial infection in a subject, by administering to the subject a therapeutically effective amount of a composition including an extracellular domain of Toll-Like Receptor 4, e.g., a fusion protein including an extracellular domain of TLR4 fused to another protein, e.g., an IgG Fc fragment, e.g., a TLR4:Fc.

In some embodiments, the subject is at risk for developing sepsis, e.g., has penetrating trauma to the abdomen, heart valve disease, and/or a large bowel incarceration. In some embodiments, the subject has one or more symptoms of sepsis, e.g., shaking, chills, fever, weakness, confusion, nausea, vomiting, and/or diarrhea. In some embodiments, the subject has one or more symptoms of septic shock, e.g., confusion and decreased consciousness; shaking chills; a rapid rise in or lower than normal temperature; warm, flushed skin; a rapid, pounding pulse; excessively rapid breathing; blood pressure that rises and falls; and/or extremities that are cool, pale, and bluish.

In another aspect, the invention provides methods for removing soluble Myeloid Differentiation Antigen-2 (sMD-2) from the blood of a subject. The methods include removing blood from the subject; contacting the blood with a TLR4:Fc fusion protein under conditions and for a time sufficient to bind sMD-2 in the blood to the TLR4:Fc, e.g., substantially all of the sMD-2, thereby forming TLR4:Fc/MD-2 complexes; removing the TLR4:Fc complexes from the blood; and optionally returning the blood to the subject, thereby removing soluble MD-2 from the blood of the subject. In some embodiments, the TLR4:Fc is bound to a collectible substrate, e.g., a bead, e.g., a magnetic bead. In some embodiments, the TLR4:Fc is bound to a column. In some embodiments, substantially all of the subject's blood is removed over time.

In a further aspect, the invention provides methods for identifying candidate compounds for the treatment of a disorder associated with a gram negative bacterial infection. The methods include providing a sample including lipopolysaccharide (LPS) and Myeloid Differentiation Antigen-2 (MD-2), e.g., soluble MD-2; contacting the sample with a test compound, e.g., a test compound that is an analog of lipid A or a portion thereof; and evaluating LPS binding to MD-2 in the presence of the test compound. A test compound that inhibits binding of LPS to MD-2 as compared to a reference, e.g., LPS binding to MD-2 in the absence of the test compound, is a candidate compound for the treatment of a disorder associated with a gram negative bacterial infection.

In some embodiments, the methods also include providing a sample including a cell expressing TLR4 that is capable of LPS-induced signalling; contacting the sample with LPS and a candidate compound that inhibits binding of LPS to MD-2; and evaluating LPS-induced signalling in the cell. A candidate compound that inhibits LPS-induced signalling in the cell is a candidate therapeutic compound for the treatment of a disorder associated with a gram negative bacterial infection.

In some embodiments, the methods also include providing an in vivo model of a disorder associated with a gram negative bacterial infection; administering a candidate therapeutic compound for the treatment of a disorder associated with a gram negative bacterial infection to the model; and evaluating an effect of the candidate therapeutic agent on a symptom of the disorder in the model. A candidate therapeutic compound that causes an improvement in a symptom of the disorder is a candidate therapeutic agent for the treatment of the disorder. The in vivo model can be, e.g., an animal infected with a gram negative bacteria, e.g., an animal other than a mouse.

The invention also provides methods for diagnosing a subject with a gram negative bacterial infection, by measuring levels of soluble Myeloid Differentiation Antigen-2 (sMD-2) in a sample from the subject, e.g., a sample including a biological fluid, e.g., blood, e.g., serum. An elevated level of sMD-2 as compared to a reference, e.g., a reference level from a healthy individual, indicates that the subject has a gram negative bacterial infection. In some embodiments, the reference level is at least 1.5 nM sMD-2, e.g., 2 nM, 3 nM, or 5 nM sMD-2 or more.

Also provided herein are additional methods of diagnosing a subject with a gram negative bacterial infection, by measuring levels of LPS in a sample from the subject, e.g., a sample including a biological fluid, e.g., blood, e.g., serum, using a competition binding assay as described herein. An elevated level of LPS as compared to a reference, e.g., a reference from a healthy individual, indicates that the subject has a gram negative bacterial infection.

Further, in another aspect the invention provides methods for detecting the presence and/or amount of LPS in a sample. The methods include providing a sample, e.g., a sample that includes a biological fluid, e.g., blood, e.g., serum, e.g., a sample suspected of containing LPS; contacting the sample with MD-2 in the presence of labeled LPS; and detecting binding of the labeled LPS to the MD-2 in the sample. An effect on binding in the sample indicates whether LPS is present in the sample, e.g., a reduction in binding as compared to a reference, e.g., a reference in the absence of the sample, indicates the presence of LPS in the sample, or a level of binding that is substantially similar to a reference, e.g., a reference in the presence of a known, selected amount of unlabelled LPS, indicates the amount of LPS in the sample.

In yet another aspect the invention provides methods for identifying candidate compounds for the treatment of a disorder associated with a gram negative bacterial infection. The methods include providing a sample including TLR4, e.g., TLR4:Fc, and MD-2; contacting the sample with a test compound; and evaluating TLR4 binding to MD-2 in the presence of the test compound. A test compound that inhibits binding of TLR4 to MD-2 as compared to a reference, e.g., TLR4 binding to MD-2 in the absence of the test compound, is a candidate compound for the treatment of a disorder associated with a gram negative bacterial infection.

These methods can also include providing a sample including a cell expressing TLR4 that is capable of LPS-induced signalling; contacting the sample with LPS and a candidate compound that inhibits binding of TLR4 to MD-2; and evaluating LPS-induced signalling in the cell. A candidate compound that inhibits LPS-induced signalling in the cell is a candidate therapeutic compound for the treatment of a disorder associated with a gram negative bacterial infection.

In some embodiments, the methods also include providing an in vivo model of a disorder associated with a gram negative bacterial infection; administering a candidate therapeutic compound for the treatment of a disorder associated with a gram negative bacterial infection to the model; and evaluating an effect of the candidate therapeutic agent on a symptom of the disorder in the model. A candidate therapeutic compound that causes an improvement in a symptom of the disorder is a candidate therapeutic agent for the treatment of the disorder. In some embodiments, the in vivo model is an animal infected with a gram negative bacteria, e.g., an animal other than a mouse.

In an additional aspect, the invention provides in silico screening methods for identifying a test compound that interacts with an MD-2 polypeptide, e.g., human MD 2 polypeptide, using a three-dimensional model of a complex including an MD-2 polypeptide bound to a ligand including lipid A to design a test compound that interacts with the MD-2 polypeptide, wherein the test compound is a lipid A analog, e.g., includes a structural analog of the disaccharide and/or acyl portions of lipid A.

In some embodiments, the three-dimensional model includes a ligand binding domain of the MD-2 polypeptide. In some embodiments, the three-dimensional model includes structural coordinates of atoms of the MD-2 polypeptide, e.g., experimentally determined coordinates.

In some embodiments, the three-dimensional model includes structural coordinates of the ligand. The methods can include altering the ligand of the model, e.g., by changing the structural coordinates of the ligand and/or by changing the chemical structure of the ligand. The changes to the ligand model can then be evaluated using methods known in the art to predict their effect, e.g., by evaluating energetic minima.

The three-dimensional model can include structural coordinates of an atom selected from the group consisting of atoms of amino acids Lys 128, Lys 132, Cys 95, and Cys 105 of the MD-2 polypeptide as defined by the amino acid positions of SEQ ID NO:2.

In some embodiments, the methods include calculating a distance between an atom of the MD-2 polypeptide and an atom of the compound.

In some embodiments, the methods include comparing a predicted interaction between the compound and the MD-2 polypeptide with the interaction between the ligand and the MD-2 polypeptide.

In some embodiments, the methods include providing a composition including an MD-2 polypeptide, and optionally a test compound that interacts with the MD-2 polypeptide, and experimentally determining an interaction of the compound with the MD-2 polypeptide, e.g., in the presence and absence of a compound including lipid A, e.g., by determining the ability of the test compound to compete for binding of LPS, e.g., labeled LPS, to MD-2. The interaction of the test compound can be compared with the MD-2 polypeptide to an interaction of a second agent, e.g., a known agonist or antagonist, with the MD-2 polypeptide.

In a further aspect, the invention provides methods for identifying a compound that interacts with an MD-2 polypeptide. The methods include designing a test compound by performing computer-aided rational drug design with a three-dimensional structure of an MD-2 polypeptide, wherein the test compound is designed to interact with a hydrophobic pocket of MD-2; contacting the test compound with an MD-2 polypeptide; and detecting the ability of the test compound to bind to the MD-2 polypeptide, e.g., by determining the ability of the test compound to compete for binding of LPS, e.g., labeled LPS, to MD-2. The methods can also include detecting an effect of the compound on TLR4 signalling, e.g., by detecting an effect on NF-κB transcriptional activity, wherein a compound that effects TLR4 signalling is a candidate compound for the treatment of a disorder characterized by TLR4 signalling. Alternatively or in addition, the methods can also include detecting an effect of the compound on an in vivo model of a disorder associated with TLR4 signalling, e.g., gram-negative infection, sepsis, septic shock, or sterile inflammation, e.g., rheumatoid arthritis, psoriasis, or Crohn's disease. In some embodiments, the test compound is selected using computer modeling. In some embodiments, the methods include synthesizing the test compound.

In another aspect, the invention includes software systems that include instructions for causing a computer system to accept and/or store information relating to the structure of an MD-2 polypeptide bound to a ligand, e.g., a ligand including lipid A; accept and/or store information relating to a test compound; and determine binding characteristics of the test compound to the MD-2 polypeptide, e.g., using energy minima. The test compound can be, e.g., a lipid A analog, e.g., include a structural analog of the disaccharide and/or acyl portions of lipid A. The determination of binding characteristics is generally based on the information relating to the structure of the MD-2 polypeptide bound to the ligand, and the information relating to the candidate agent.

Also provided herein are software programs residing on a machine readable medium having a plurality of instructions stored thereon, which, when executed by one or more processors, cause the one or more processors to accept information relating to the structure of a complex including an MD-2 polypeptide bound to a ligand, e.g., a ligand including lipid A; accept information relating to a test compound; and determine binding characteristics of the test compound to the MD-2 polypeptide, wherein the test compound is a lipid A analog, e.g., is or includes a structural analog of the disaccharide and/or acyl portions of lipid A, and wherein the determination of binding characteristics is based on the information relating to the structure of the MD-2 polypeptide and the information relating to the test compound.

Further, the invention provides methods for optimizing the structure of a test compound that interacts with an MD-2 polypeptide. The methods include accepting information relating to the structure of a complex including an MD-2 polypeptide bound to a ligand; and modeling the binding characteristics of the MD-2 polypeptide with a test compound, wherein the test compound is a lipid A analog, e.g., is or includes a structural analog of the disaccharide and/or acyl portions of lipid A, and optimizing the structure of the test compound to enhance binding to the MD-2 polypeptide, wherein the method is implemented by a software system.

Kits for detecting the presence and/or level of LPS in a sample including MD-2, e.g., MD-2 bound to a solid surface such as a slide; directly or indirectly labeled LPS (or an MD-2 binding portion thereof, e.g., lipid A), e.g., a known quantity of LPS; reagents for detecting binding of the LPS to the MD-2, e.g., avidin-HRP, if the LPS is biotinylated; and optionally, a reference, e.g., a reference that represents a selected level of endotoxin (e.g., a level of endotoxin above which a tested sample is not usable, or a level above which a gram negative bacterial infection is diagnosed) or a number of references (e.g., to allow quantification of the level of endotoxin present in the sample) are also provided herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein, including sequence accession numbers, are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a graph showing the results of Fluorescence Activated Cell Sorting (FACS) analysis of MD-2 binding to living S. typhimurium cells.

FIG. 2A is a Western blot showing secreted N-FLAG tagged MD-2 from the supernatants of stably transduced HEK293 cells, immunoprecipitated with an anti-FLAG monoclonal antibody (lane 1), or precipitated with biotin-LPS using streptavidin beads (SAB, lane 2 and 3). Note that only the monomeric form of MD-2 binds to biotinylated LPS.

FIG. 2B is a Western blot showing TLR4^{YFP} immunoprecipitated using a polyclonal anti-GFP antibody, separated by SDS-PAGE under non reducing conditions, and detected by anti-biotin western blotting. The 160 kDa band corresponds to surface TLR4, while the 25 kDa protein corresponds to the co-precipitated MD-2. LPS treatment neither affected binding of MD-2 to TLR4 nor altered the aggregation status of MD-2 on the cell surface (lane 2).

FIG. 3A is a Western blot showing TLR4:Fc (top panel) and MD-2 (bottom panel) proteins from conditioned media from MD-2 and TLR4:Fc expressing cells were mixed in equal amounts (lanes 3-5) and captured with streptavidin beads in the presence (lanes 1, 3) or absence (lane 2) of biotinylated LPS (1 μg/ml). Samples were subjected to biotin-LPS precipitation (lanes 1, 3) or protein A precipitation (lanes 4-6). The blots were probed with HRP-labeled anti-mouse polyclonal Ab (for the TLR4:Fc chimera, upper portion of the membrane) or an anti-FLAG mAb (for MD-2^{FLAG}, bottom portion of the membrane).

FIG. 3B is a Western blot illustrating that MD-2 and TLR4 bind to LPS in a 1:1 ratio. Lysates from cells expressing both FLAG-tagged TLR4 and FLAG-tagged MD-2 were incubated with streptavidin beads and the bound proteins were analyzed by western blotting with anti-FLAG mAb. TLR4^{FLAG} and MD-2^{FLAG} were also immunoprecipitated with an anti-TLR4 monoclonal antibody (HTA125) or an anti-FLAG mAb, respectively (lanes 4 and 6) as controls. The blot was probed with an anti-FLAG antibody. The TLR4 and MD-2 intensities correlate with their relative amounts in the lysates.

FIG. 4A is a line graph of MD-2^{6xHis} binding to TLR-4:Fc. MD-2^{6xHis} was added in titrated amounts (0.1 to 50 nM). To control for non-specific binding to protein A, 50 nM of MD-2 was plated in the absence of TLR4:Fc (“50”). The dotted line shows the averages of triplicate absorbance readings taken at 450 nm±SD. The apparent kDa of this interaction is of ˜12 nM. To determine the effect of LPS on the affinity of MD-2 for TLR4, the binding experiments were also performed in the presence of 0.1 μg of LPS/ml (solid line).

FIG. 4B is a line graph of MD-2^{6×His} binding to TLR-4:Fc. TLR4:Fc captured on protein A coated plastic at the indicated concentrations (1.1, 2.2, 4.4, 8.8, 17.5, or 35 nM) and MD-2 (12 nM) was added without (filled circles) or with LPS (0.1, 1, or 10 μg/ml, open symbols). The background of the Ni-HRP reagent on the titrated TLR4:Fc, in the absence of added MD-2, is shown by the scattered line.

FIG. 5A is a 3-D bar graph showing NF-κB activation in HEK293 cells stably expressing TLR4^{YFP} and MD-2^{FLAG}, transiently transfected with a NF-kB luciferase reporter plasmid. The cells were then stimulated with increasing amounts of LPS (x axis, from right to left) in the presence of increasing amounts of the LPS antagonist E5564 (y axis, dark to light bars). Luciferase activity was measured using a multiplate luminometer.

FIG. 5B is a Western blot showing TLR4^{YFP} detected with an anti-GFP mAb, in lysates from cells treated with biotin-LPS (0.5 μg/ml) for one hour at 37° C. in the absence (lane 1) or presence (lanes 2-5) of increasing amounts of the LPS antagonist E5564.

FIG. 5C is a Western blot showing the results of a similar experiment to the one shown in 5B, performed by adding biotin-LPS plus variable amounts of E5564 to conditioned medium containing soluble MD-2 (10 ml/lane); sMD-2 was precipitated with streptavidin beads and analyzed by western blot with an anti-FLAG mAb as in FIG. 3A.

FIG. 5D is a Western blot showing that binding of biotin-LPS to soluble MD-2 (lane 1) could be abrogated using a tenfold excess (w/v) of non labeled LPS (lane 2), E5564 (lane 3) or the synthetic TLR4 agonist, ER112022 (lane 4).

FIG. 6A is a line graph showing soluble MD-2-activity depleted from human healthy serum using a TLR4:Fc chimera. 293 cells stably expressing TLR4^{YFP} were transiently transfected with an NF-κB-luciferase reporter plasmid and treated overnight with increasing amounts of LPS in 20% human serum that had been pretreated with protein A beads (PAS, scattered line), TLR2:Fc loaded PAS (triangles) or TLR4:Fc loaded PAS (circles).

FIG. 6B is a line graph showing MD-2 depleted serum (circles, same as in A) was reconstituted with 60 nM MD-2 and used in the stimulation assay (squares). Results are shown as average of duplicate luciferase readings divided by the untreated point (0, no LPS)±SD. Note that the experiment shown in A and B is representative on one of three experiments, each performed with a different human volunteer.

FIG. 6C is a pair of line graphs showing the results pf an experiment in which human serum was depleted of MD-2 using TLR4:Fc (open squares) or mock depleted (PAS only, open circles) and used to stimulate TLR4/NF-kB-Luciferase reporter cells with increasing amounts of LPS (left portion of the graph). MD-2 depleted serum was then reconstituted with increasing amounts of soluble purified recombinant MD-2 (right portion of the graph) at four different concentration of LPS (500 ng/ml, filled squares, 100 ng/ml filled circles, 50 ng/ml triangles and 10 ng/ml diamonds). The activation conferred by 50 ng of LPS/ml in 20% mock treated serum is indicated by the double headed arrow. Results are shown as the average luciferase units of duplicate readings±SD.

FIG. 7A is a line graph showing the results pf an experiment in which cells expressing TLR4^{YFP} and an NF-κB luciferase reporter plasmid were stimulated with increasing concentrations of LPS in the absence (diamonds) or the presence of TLR4:Fc at the indicated concentrations. Shown is the average of duplicate luciferase reading±SD.

FIG. 7B is a Western blot of the same cells used in 7A, after treatment with 1 μg of biotin-LPS/ml in the absence (lanes 2 and 3) or in the presence of TLR4:Fc (lanes 3-5). As a control, the maximum amount of TLR4:Fc was added to the cells in the absence of biotin-LPS. Note that the presence of TLR4:Fc prevented the interaction of biotinylated LPS with cellular MD-2.

FIGS. 7C and D are line graphs showing release of IL-6 was measured by ELISA from adherent human PBMC, treated as in 7A, in the absence (7C) or in the presence (7D) of 60% autologous human serum for 4 hours. Shown are the averages of absorbance units±SD.

FIGS. 8A-8C are schematic illustrations of the chemical structures of lipid A as found in E. coli strains (8A); LPS agonist ER-112022 (8B); and LPS antagonist E5564 (8C).

FIG. 9 is a computer-generated theoretical ribbon model of the structure of MD-2. Lysines 128 and 132 are shown in space-filling mode, as is the D loop delimited by Cysteines 95 and 105; this loop is thought to be important for signaling.

FIG. 10 is a space filling model showing the empirically-determined crystal structure of LPS lipid A.

FIGS. 11 and 12 are two side views of lipid A moiety (space filling model) docked in the hydrophobic pocket of MD-2 (ribbon model).

FIGS. 13, 14, and 15 are top-down views of a theoretical ribbon model of the structure of MD-2 (FIG. 13); the LPS lipid A moiety (space filling model) docked in the hydrophobic pocket of MD-2 (ribbon model) (FIG. 14); and a space filling model showing the empirically-determined crystal structure of LPS lipid A (FIG. 15).

### DETAILED DESCRIPTION

The syndrome of Gram-negative sepsis has long been studied as a disease whose pathogenesis is thought to be due to the toxic effects of LPS. Although formal proof of this association has never been established, the circumstantial evidence that LPS causes the initial toxicity associated with a deeply invasive Gram-negative infection is overwhelming. In part, the lack of formal proof is related to the essential nature of LPS. Only a single mutant Gram-negative bacterium that is entirely lacking LPS has been engineered, and in an organism for which no good animal model exists (N. meningitiditis (31)). Nevertheless, there are numerous published reports relating the effects of endotoxin to sepsis, and Gram-negative organisms that express attenuated endotoxins are less proinflammatory (see, e.g., (32)). Certainly, of all of the immune modulating molecules expressed by Gram-negative organisms, endotoxin is the most potent initiator of proinflammatory events.

Faced with this circumstantial evidence, investigators and pharmaceutical companies have long desired to identify molecules that might be used therapeutically for sepsis, and perhaps for other diseases said to be due to endotoxin. Many such molecules have been identified, including LPS neutralizing proteins and peptides, although the value of such molecules to patients remains to be proved. One relatively newer category of anti-endotoxin agents are the lipid A-based LPS inhibitors. These analogs of toxic lipid A have previously been thought to be LPS receptor antagonists. The mechanism of action of these agents could not be defined, because until relatively recently, the LPS receptor was an undefined, hypothetical entity. With the identification of LBP and CD14, there was initial optimism that either molecule might be their target. This proved to be incorrect, because both LBP and CD14 are simply LPS-enhancing proteins (albeit potent ones) that work together with MD-2 on the surface of bacteria to bring the LPS present in the outer leaflet of the outer membrane to the TLR4 signal transducer (4). The essence of the difference between TLR4/MD-2 and LBP/CD14 is that the latter two molecules are not absolutely required for LPS responses.

In contrast, both TLR4 and MD-2 appear to be essential for cells to respond to LPS, at least with the respect to the induced production of the immune mediators that are associated with the sepsis syndrome. The minimal composition of the LPS receptor unit was explored in detail. The results, described herein, demonstrated that successful binding of LPS to its signaling receptor does not require other factors of cellular origin, except for MD-2, which can be provided, and exists in serum, as a soluble molecule. In the soluble phase, the ectodomain of TLR4, MD-2 and LPS form a stable complex, with an apparent K_{d} for TLR4/MD-2 interactions of 12 nM. Accordingly, TLR4 positive human cells could be efficiently triggered (i.e., activate TLR4 signalling, resulting in NF-kB activation) under protein free conditions by supplementing the serum with less than 1 nM MD-2 in the presence of LPS; activation levels were proportional to the concentration of sMD-2. The relative importance of CD14 and LBP and the absolute importance of MD-2 in LPS responses are in accordance with the data previously reported for the CD14, LBP and MD-2 knock out mice (7, 20-22).

The importance of LPS binding to MD-2 was highlighted by the discovery, described herein, that LPS-inhibitory lipid A analogs, such as the synthetic compound E5564, appear to function by preventing LPS/MD-2 interactions. Moreover, data described herein support the hypothesis that monomeric MD-2 is the only physiologically relevant species of the molecule, as only monomeric MD-2 interacts with LPS or TLR4 on the cell surface. Finally, using soluble TLR4:Fc fusion proteins as a probe, it was found that normal “healthy” human serum contains about 1.5 nM of functional soluble MD-2. Soluble MD-2 is capable of binding to living bacteria, suggesting a physiological role for soluble MD-2 as an active sentinel for the innate immune system. Furthermore, these results indicate that you can inhibit LPS responses by “sequestrating” or “inactivating” soluble MD-2. Such an approach is attractive, since there is likely to be less MD-2 present than LPS (in a cohort of patients with meningococcal systemic infection, the median LPS was 380 pg/ml), and the Kd for the interaction (12 nM) is higher than the concentration of MD-2, which suggests that most of TLR4 is not ligated with MD-2 in the serum.

The finding that the drug target for the lipid A analogs is MD-2 is surprising, as molecular genetic studies in humans and mice with pharmacological antagonists such as lipid IVa (a lipid A precursor) suggested that this class of drugs would primarily function by interacting with TLR4 (33). Lipid IVa is an LPS agonist in mice, but an antagonist in humans. While there may, in fact, be an interaction of compounds such as lipid IVa with TLR4, the results described herein leave little doubt that compounds such as lipid IVa, including E5564, inhibit LPS signaling primarily by interfering with LPS binding to MD-2.

In addition to small molecules that interfere with LPS binding to MD-2, the results described herein demonstrate that one can also interrupt LPS signaling by blocking the binding of MD-2 to cell-associated TLR4. The use of a TLR4:Fc fusion protein is described herein, but other compounds, e.g., small molecules or monoclonal antibodies, that block MD-2/TLR4 interactions, should have substantially the same effects. Subjects at the highest risk for developing sepsis, such as those with penetrating trauma to the abdomen or large bowel incarceration, would be ideal candidates for prophylaxis with agents that inhibit MD-2 function.

The results described herein support a model of cellular activation that indicates that MD-2 likely undergoes an LPS-dependent conformational change that in turn induces the homotypic aggregation of TLR4/MD-2, followed by the recruitment of MyD88 and, presumably, the other adapter molecules.

Methods of Screening

Also included herein are methods for screening test compounds, e.g., lipids, polypeptides, polynucleotides, inorganic or organic large or small molecule test compounds, to identify agents useful in the treatment of disorders associated with infection with Gram-negative organisms, e.g., sepsis and septic shock, that are analogs of lipid A or a portion thereof. The methods include using rational drug design methods to identify structures that are similar to lipid A or a portion thereof, and screen them for the ability to interfere with binding of LPS and MD-2, or with binding of TLR4 and MD-2.

Included herein are methods (also referred to herein as “screening assays”) for identifying modulators, i.e., test compounds or agents (e.g., proteins, peptides, peptidomimetics, peptoids, small molecules or other drugs) that decrease, e.g., inhibit or prevent, MD-2, e.g., sMD-2, binding to LPS or TLR4. This can be accomplished, for example, by coupling one of MD-2, LPS, or TLR4 with a label, e.g., a radioisotope or non-isotopic label, such that binding of MD-2 to LPS or TLR4 can be determined by detecting the labeled compound in a complex. Alternatively, MD-2, TLR4 and/or LPS can be coupled with a radioisotope or enzymatic label to monitor the ability of a test compound to modulate MD-2 binding to LPS in a complex. For example, LPS, TLR4 and/or MD-2 can be labeled with ¹²⁵I, ³⁵S, ¹⁴C, or ³H, either directly or indirectly, and the radioisotope detected by direct counting of radioemission or by scintillation counting. Alternatively, compounds can be directly or indirectly enzymatically labeled with, for example, biotin, horseradish peroxidase, alkaline phosphatase, or luciferase, and the enzymatic label detected by determination of conversion of an appropriate substrate to product. For example, biotin-LPS can be detected using an avidin-HRP stain. See, e.g., Visintin et al., J Biol Chem 278:48313 (2003).

The ability of MD-2 to interact with LPS or TLR4 with or without the labeling of any of the interactants can be evaluated. For example, a microphysiometer can be used to detect the interaction of MD-2 and LPS or TLR4 without the labeling of MD-2, TLR4 or LPS. See, e.g., McConnell et al., Science 257:1906-1912 (1992). As used herein, a “microphysiometer” (e.g., Cytosensor®, Molecular Devices Corporation, Sunnyvale Calif.) is an analytical instrument that measures the rate at which a cell acidifies its environment using a light-addressable potentiometric sensor (LAPS). Changes in this acidification rate can be used as an indicator of the interaction between MD-2 and LPS or TLR4.

Soluble forms of MD-2 (sMD-2) and/or TLR4 proteins, or biologically active portions thereof, will generally be used in the assays described herein. For example, a soluble form of TLR4 can include all or part of the extracellular domain, e.g., amino acids 1-631 or 632 of the human TLR4 (GenBank Accession No. NP_{—}612564.1; SEQ ID NO:1), with or without amino acids 1-23, which are the signal peptide (e.g., including only amino acids 24-631 or 632), or can include a TLR4:Fc fusion protein as described herein.

NP_612564 839 aa linear PRI 02-APR-2006
Toll-Like Receptor 4 Precursor [Homo sapiens]-
SEQ ID NO:1

| | | | | |
|---|---|---|---|---|
| 1 | MMSASRLAGT | LIPAMAFLSC | VRPESWEPCV | EVVPNITYQC |
| | MELNFYKIPD | NLPFSTKNLD | | |
| 61 | LSFNPLRHLG | SYSFFSFPEL | QVLDLSRCEI | QTIEDGAYQS |
| | LSHLSTLILT | GNPIQSLALG | | |
| 121 | AFSGLSSLQK | LVAVETNLAS | LENFPIGHLK | TLKELNVAHN |
| | LIQSFKLPEY | FSNLTNLEHL | | |
| 181 | DLSSNKIQSI | YCTDLRVLHQ | MPLLNLSLDL | SLNPMNFIQP |
| | GAFKEIRLHK | LTLRNNFDSL | | |
| 241 | NVMKTCIQGL | AGLEVHRLVL | GEFRNEGNLE | KFDKSALEGL |
| | CNLTIEEFRL | AYLDYYLDDI | | |
| 301 | IDLFNCLTNV | SSFSLVSVTI | ERVKDFSYNF | GWQHLELVNC |
| | KFGQFPTLKL | KSLKRLTFTS | | |
| 361 | NKGGNAFSEV | DLPSLEFLDL | SRNGLSFKGC | CSQSDFGTTS |
| | LKYLDLSFNG | VITMSSNFLG | | |
| 421 | LEQLEHLDFQ | HSNLKQMSEF | SVFLSLRNLI | YLDISHTHTR |
| | VAFNGIFNGL | SSLEVLKMAG | | |
| 481 | NSFQENFLPD | IFTELRNLTF | LDLSQCQLEQ | LSPTAFNSLS |
| | SLQVLNMSHN | NFFSLDTFPY | | |
| 541 | KCLNSLQVLD | YSLNHIMTSK | KQELQHFPSS | LAFLNLTQND |
| | FACTCEHQSF | LQWIKDQRQL | | |
| 601 | LVEVERMECA | TPSDKQGMPV | LSLNITCQMN | KTIIGVSVLS |
| | VLVVSVVAVL | VYKFYFHLML | | |
| 661 | LAGCIKYGRG | ENIYDAFVIY | SSQDEDWVRN | ELVKNLEEGV |
| | PPFQLCLHYR | DFIPGVAIAA | | |
| 721 | NIIHEGFHKS | RKVIVVVSQH | FIQSRWCIFE | YEIAQTWQFL |
| | SSRAGIIFIV | LQKVEKTLLR | | |
| 781 | QQVELYRLLS | RNTYLEWEDS | VLGRHIFWRR | LRKALLDGKS |
| | WNPEGTVGTG | CNWQEATSI | | |

When less-soluble or non-soluble species are used (e.g., lipid A), it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, isotridecypoly(ethylene glycol ether)n, 3-[(3-cholamidopropyl) dimethylamminio]-1-propane sulfonate (CHAPS), 3-[(3-cholamidopropyl) dimethylamminio]-2-hydroxy-1-propane sulfonate (CHAPSO), or N-dodecyl=N,N-dimethyl-3-ammonio-1-propane sulfonate.

In some embodiments, the assay is carried out in a defined solution containing human serum, human serum albumin, or other serum components, e.g., LBP and CD14. sMD-2 is generally readily soluble in saline or serum.

In some embodiments, the methods described herein include applying a test compound to a test sample including a cell or living tissue or organ, and evaluating one or more effects of the test compound, e.g., the ability of the test compound to disrupt LPS-activated signaling.

In some embodiments, the test sample is, or is derived from (e.g., a sample originally taken from) an in vivo model of a disorder as described herein. For example, an animal model, e.g., a rodent such as a rat, that is infected with a gram negative bacterium can be used, and the ability of the test compound to improve one or more symptoms of the disorder, e.g., clinically relevant symptoms, are evaluated.

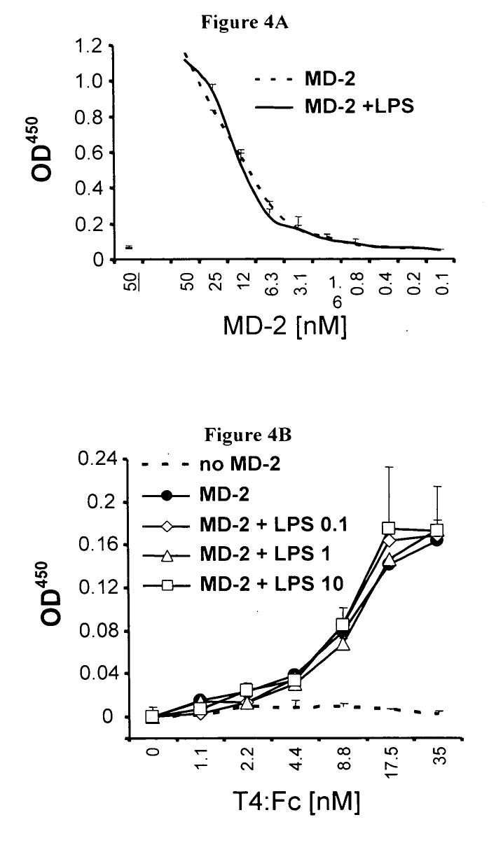

Methods for evaluating each of these effects are known in the art; some are described herein. For example, an ELISA, e.g., as described in Example 5 and illustrated in FIGS. 4A and 4B can be used to screen for molecules that interfere with TLR4/MD-2 binding in vitro.

A test compound that has been screened by a method described herein and determined to interfere with LPS/sMD-2 or sMD2/TLR4 binding, and to interfere with LPS signalling in a TLR4-expressing cell, can be considered a candidate compound. A candidate compound that has been screened, e.g., in an in vivo model of a disorder, e.g., an animal infected with a gram negative bacterium or administered a dose of LPS, and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified by a method described herein as candidate therapeutic compounds can be further screened by administration to an animal model of a disorder associated with TLR4-signalling, e.g., a model of gram negative infection or of sterile inflammation, as described herein. Test compounds identified as hits can be considered candidate therapeutic compounds, useful in treating these disorders. A variety of techniques useful for determining the structures of “hits” of unknown structure, e.g., a compound in a library, can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Compounds that interfere with binding of LPS and MD-2 or LPS and TLR4 can be identified using, e.g., cell-based or cell free assays, as are known in the art. Such compounds can also be further screened in animal models.

Cell-Free Assays

Cell-free assays typically involve preparing a reaction mixture of the target gene protein and the test compound under conditions and for a time sufficient to allow the two components to interact and bind, forming a complex that can be removed and/or detected. A number of suitable TLR-ligand binding assays, including FRET, LANCE, alpha assays, and others, are described in U.S. patent application Ser. No. 11/014,351, filed Dec. 16, 2004, U.S. Pat. App. Pub. No. US 2005-0208470 the entire contents of which are incorporated herein by reference.

The interaction between two molecules can also be detected, e.g., using fluorescence energy transfer (FET) (see, for example, Lakowicz et al., U.S. Pat. No. 5,631,169; Stavrianopoulos et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor.’ Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FET binding event can be conveniently measured through standard fluorimetric detection means well known in the art (e.g., using a fluorimeter).

In another embodiment, determining the ability of MD-2 to bind to LPS or TLR4 can be accomplished using real-time Biomolecular Interaction Analysis (BIA) (see, e.g., Sjolander and Urbaniczky, (1991) Anal. Chem. 63:2338-2345; Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In some embodiments, either MD-2, LPS or TLR4 is anchored onto a solid phase. The MD-2/LPS or MD-2/TLR4 complexes anchored on the solid phase can be detected at the end of the reaction. For example, MD-2 can be anchored onto a solid surface, and LPS, (which is not anchored), can be labeled, either directly or indirectly, with detectable labels discussed herein. Alternatively, TLR4, e.g., TLR4:Fc, can be anchored, and MD-2 can be labeled.

In some embodiments, the assay is an Enzyme Linked Immuno-Sorbent Assay (ELISA), e.g., a biotin-LPS displacement assay. Such assays have the advantage of being generally cheap, fast and automatable. For example, MD-2 can be immobilized on plastic, and binding of biotin-LPS can be detected using an avidin-HRP stain, or TLR4 (e.g., TLR4:Fc) can be immobilized, and binding of MD-2 (e.g., biotinylated or otherwise labeled, e.g., fluorescent) can be detected. Test compounds can be assayed to see if they affect binding, e.g., if binding of biotin-LPS or fluorescent-MD2 can no longer be detected or is significantly reduced.

Thus, it may be desirable to immobilize MD-2, TLR4 or LPS to facilitate separation of complexed from uncomplexed forms of one or both of the proteins, as well as to accommodate automation of the assay. Binding of MD-2 to LPS or of MD-2 to TLR4, e.g., in the presence and absence of a candidate compound, can be accomplished in any vessel suitable for containing the reactants. Examples of such vessels include microtiter plates, test tubes, and micro-centrifuge tubes.

In some embodiments, a fusion protein can be used that adds a domain that allows the MD-2 or TLR4 protein to be, e.g., bound to a matrix. For example, glutathione-S-transferase/MD-2 fusion proteins can be adsorbed onto glutathione Sepharose™ beads (Sigma Chemical, St. Louis, Mo.) or glutathione derivatized microtiter plates. Alternatively, a TLR4:Fc fusion protein, e.g., as described herein, can be adsorbed onto protein A-coated surface, e.g., beads or plates. The coated surfaces can then be combined with a test compound, or a test compound and either the non-adsorbed MD-2, TLR4, or LPS, and the mixture incubated under conditions conducive to complex formation (e.g., at physiological conditions for salt and pH as described herein). Following incubation, the beads or microtiter plate wells are washed to remove any unbound components, the matrix immobilized in the case of beads, and the presence of complexes determined either directly or indirectly, for example, as described above. Alternatively, the complexes can be dissociated from the matrix, and the level of MD-2/LPS or MD-2/TLR4 binding can be determined using known techniques.

Other techniques for immobilizing MD-2, TLR4, and/or LPS on matrices include using conjugation of biotin and streptavidin. Biotinylated MD-2, TLR4, and/or LPS can be prepared from biotin-NHS (N-hydroxy-succinimide) using techniques known in the art (e.g., using biotinylation kits available from Pierce Chemicals, Rockford, Ill.), and immobilized in the wells of streptavidin-coated 96 well plates (Pierce Chemical).

High protein binding plastic substrates can also be used; the species to be immobilized is simply adsorbed to the plastic. These substrates do not require any modification of the species to be immobilized. Suitable substrates are commercially available and include multi-well plates, e.g., Microlon® ELISA 96-well Immunoassay Plates (Bellco Glass, Inc., Vineland, N.J.), EIA/RIA Immunoassay Plates (E&K Scientific, Campbell, Calif.).

To conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways known in the art. For example, where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody). In embodiments were the LPS is biotinylated, detection can be with avidin-HRP.

In some embodiments, this assay is performed utilizing TLR4, MD-2- or LPS-specific binding proteins, e.g., TLR4:Fc (which binds MD-2, as described herein), anti-MD-2 antibodies, anti-TLR4 antibodies, or anti-LPS antibodies, but that do not interfere with binding of MD-2 to LPS, or of MD-2 to TLR4, depending on which is being assayed. Such specific binding proteins can be derivatized to a surface, e.g., beads or the wells of a plate, and unbound MD-2, TLR4, or LPS trapped in the wells by antibody conjugation. Methods for detecting such complexes, in addition to those described above for the GST- or protein-A immobilized complexes, include immunodetection of complexes using antibodies reactive with the MD-2, TLR4, or LPS, as well as enzyme-linked assays that rely on detecting an enzymatic activity associated with the MD-2, TLR4, or LPS.

Alternatively, cell free assays can be conducted in a liquid phase. Generally, in such an assay, the reaction products are separated from unreacted components, by any of a number of standard techniques, including but not limited to: differential centrifugation (see, for example, Rivas and Minton, Trends Biochem Sci 18:284-7 (1993)); chromatography (gel filtration chromatography, ion-exchange chromatography); electrophoresis (see, e.g., Ausubel et al., eds., Current Protocols in Molecular Biology (J. Wiley: New York, 1999); and immunoprecipitation (see, for example, Id.). Suitable resins and chromatographic techniques are known to one skilled in the art (see, e.g., Heegaard, J Mol Recognit 11: 141-8 (1998); Hage and Tweed, J Chromatogr B Biomed Sci Appl. 699:499-525 (1997)). Further, fluorescence energy transfer can also be conveniently utilized, as described herein, to detect binding without further purification of the complex from solution. In some embodiments, an LPS binding assay is conducted as described in Visintin et al., J Biol Chem 278:48313 (2003).

In some embodiments, the assay is an Enzyme Linked Immmuno-Sorbent Assay (ELISA). Such assays have the advantage of being generally cheap, fast and automatable.

In one example, the assay is a biotin-LPS displacement ELISA, and the test sample can include MD-2 and biotinylated-LPS in solution; the sample can be incubated in the presence of a test compound; a control sample can include no test compound, and/or a compound that is known to interfere with binding of MD-2 and LPS (e.g., an LPS agonist or antagonist such as ER-112202 or E5564). The samples can be contacted with avidin-coated beads, and centrifuged. The resulting pellet can be analyzed using gel electrophoresis, and binding of LPS to MD-2 can be detected using an avidin-HRP stain. Compounds that inhibit binding of LPS to MD-2 will cause a decrease in the amount of staining; see, e.g., Example 6, below. Test compounds can be assayed to see if they affect LPS/MD-2binding, e.g., if binding of biotin-LPS can no longer be detected or is significantly reduced. In some embodiments, the samples also include other elements of serum, e.g., human serum, such as proteins, e.g., human serum albumin.

Alternatively or in addition, a test compound can be screened to determine if it affects the Kd of the interaction between MD-2 and TLR4, e.g., as described in Example 5. For example, a test sample including TLR4, e.g., TLR4:Fc, can be incubated in the presence of a test compound; a control sample can include no test compound, and/or a compound that is known to interfere with binding of MD-2 and TLR4. Where the test sample includes TLR4:Fc, the sample can be contacted with protein A coated surface, e.g., beads or the surface of a plate. The surface can be used to collect the TLR4/MD-2 complexes (e.g., by centrifugation in the case of beads), and binding can be detected. For example, a 6×His tagged MD-2 can be used, and binding can be detected using Ni-HRP, by measuring the absorbance of each sample at 450 nm.

To identify compounds that interfere with an interaction between MD-2 and LPS or MD-2 and TLR4, a reaction mixture containing MD-2 and LPS or MD-2 and TLR4 is prepared, and incubated under conditions and for a time sufficient, to allow the two products to form complex. To test an inhibitory agent, the reaction mixture is analyzed in the presence and absence of the test compound. The test compound can be initially included in the reaction mixture, or can be added at a time subsequent to the addition of the target gene and its cellular or extracellular binding partner. Control reaction mixtures are typically incubated without the test compound or with a negative control, or with a positive control compound known to interfere with the interaction between MD-2 and TLR4, or between MD-2 and LPS, e.g., lipid A. The formation of any complexes between MD-2 and LPS or MD-2 and TLR4 is then detected. The formation of a complex in the control reaction, but not in the reaction mixture containing the test compound, indicates that the compound interferes with the interaction of the target gene product and the interactive binding partner.

In an alternate embodiment of the invention, a homogeneous assay can be used in which a preformed complex of the MD-2 and LPS or MD-2 and TLR4 is prepared, in which either LPS, TLR4, or MD-2 is labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

Cell-Based Assays

The assays described herein can also be performed in samples including cells. Cell-based assays are known in the art, and can be used, e.g., to detect LPS/MD-2 or MD-2/TLR4 binding and/or effects that occur downstream, e.g., in a cell expressing cell-surface TLR4, whether a test compound affects a downstream effect of LPS-induced signalling. For example, a cell that expresses TLR4 can be used, and FACS analysis can be used to detect changes in binding of labeled MD-2; MD-2 can be directly conjugated with a fluorochrome or it can be detected using an antitag (e.g., anti-FLAG) antibody or similar chemistry. Molecules that interfere with binding should give lower fluorescence intensities in the histograms.

If the cells expressing TLR4 on their surface are plated on plastic, sMD-2 can be added to them (with or without a test compound or known inhibitor) and then the cells are washed, dried on plastic and tested for the presence of MD-2, e.g., by detecting a tag on the MD-2 (this is sometimes referred to as a cell based ELISA, and can be performed using commercially available kits, e.g., the Fast Activated Cell-based ELISA (FACE™) Kits, Active Motif, Inc., Carlsbad, Calif.).

In another example, cells expressing TLR4 on their surface are contacted with sMD-2 in the presence and/or absence of a test compound, for a time and under conditions sufficient to allow the formation of sMD-2/TLR4 complexes. The surface proteins can be biotinylated, and the cells then lysed. Immunoprecipitation can be used to detect the bound MD-2. Alternatively, biotinylated LPS can be used to detect the presence of the bound MD-2. Avidin can be used to detect biotinylated LPS in the FACS or ELISA based assays.

In some embodiments, the methods can include screening a test compound in a first, e.g., cell-free, assay, to identify compounds that can inhibit binding of LPS to sMD-2, or of sMD-2 to cell-surface TLR4, and then in a second, e.g., cell-based, assay, to identify those compounds that inhibit the downstream effects of LPS-induced signalling, e.g., activation of NF-κB by LPS in the presence of sMD-2. In some embodiments, cellular activation is monitored using FACS to follow upregulation of costimulatory molecules, such as ICAM or CD83, CD80, or CD86, depending on the cell type used. In some embodiments, these methods are performed using cells that express TLR4 on the surface, but not MD-2, e.g., as known in the art.

In cell-based assays of binding of MD-2 to cell-surface TLR4, the TLR4:Fc as described herein can be used as a control, as it interferes with binding between MD-2 and cell-surface TLR4, as described herein.

Animal Models

Also included herein are methods of screening compounds by administering a compound, e.g., a compound identified in a cell-free or cell-based screen as described herein as a compound that can inhibit binding of LPS to sMD-2, or of MD-2 to TLR4, e.g., compounds that can inhibit the downstream effects of LPS-induced signalling, to an animal model of gram-negative infection. Suitable animal models are known in the art, e.g., mammals infected with a gram-negative bacterium such as Escherichia coli, Helicobacter pylori, or mammals administered a sub-lethal dose of purified LPS. In some embodiments, the animal is a model of gram-negative induced septic shock. The methods include administering at least one dose of a compound to the animal, and monitoring the animal for an effect of the compound on the disorder in the animal, e.g., an effect on a clinically relevant parameter, e.g., a parameter that is related to a clinical symptom of the disease as described herein. Methods for selecting, evaluating and scoring such parameters are known in the art. In some embodiments, where the animal is given a sub-lethal dose of purified LPS, the animal is evaluated to see if administering a test compound, or a T4:Fc chimeric protein, rescues the animal.

The animal can be monitored for a change in the disorder, e.g., for an improvement in a parameter of the disorder, e.g., a parameter related to clinical outcome. In some embodiments, the parameter is fever (a trend towards or a return to normal, e.g., a decrease, would be an improvement); blood pressure (a return to normal, e.g., an increase, would be an improvement); heart rate (a trend towards or a return to normal, e.g., a decrease, would be an improvement); and respiration rate (a trend towards or a return to normal, e.g., a decrease, would be an improvement); levels of white blood cells (a trend towards or a return to normal would be an improvement); the level of oxygen (a trend towards or a return to normal, e.g., an increase, would be an improvement); the number of platelets (a trend towards or a return to normal, e.g., an increase, would be an improvement); lactic acid levels (a trend towards or a return to normal, e.g., a decrease, would be an improvement); and levels of metabolic waste products (a trend towards or a return to normal, e.g., a decrease, would be an improvement).

Test Compounds and Rational Drug Design

The methods described herein include screening test compounds and libraries thereof to identify compounds that interfere with binding of MD-2 to TLR4 or of LPS to MD-2.

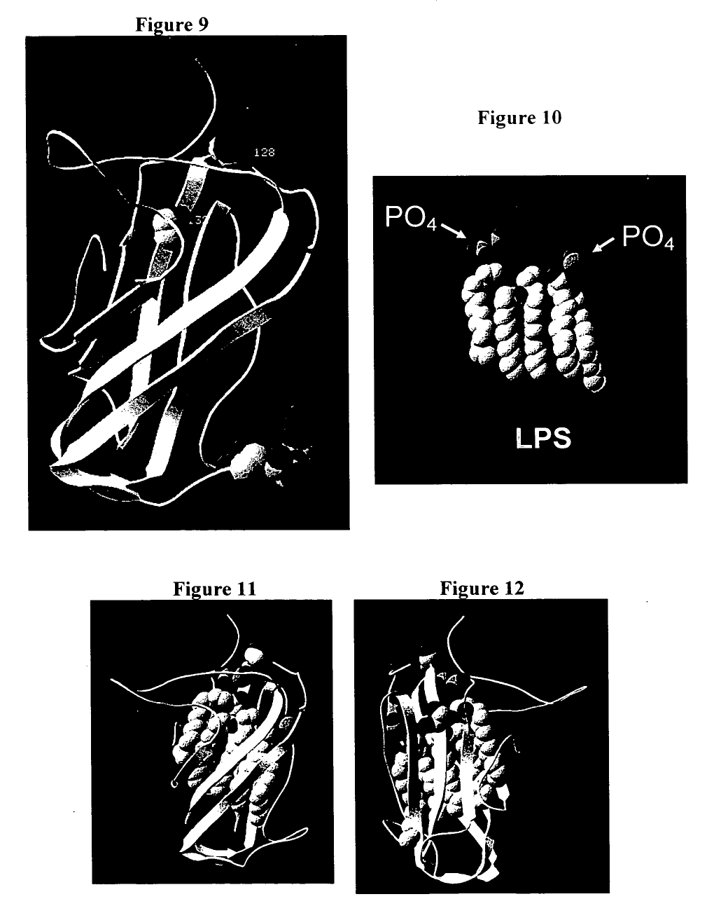

In some embodiments, the test compounds are structural analogs, e.g., small molecule analogs, of lipid A or a portion thereof, e.g., a portion that is believed to be important in LPS-binding to MD-2, such as the two phosphate groups on the disaccharide portion of the molecule, or the acyl group tails (see FIG. 10). In some embodiments, one or more of the test compounds is obtained by systematically altering the structure of a structural analog of lipid A, e.g., using methods known in the art or the methods described herein, and correlating that structure to the ability to interfere with MD-2/LPS binding, e.g., a structure-activity relationship study. As one of skill in the art will appreciate, there are a variety of standard methods for creating such a structure-activity relationship. Generally, the three-dimensional structure of lipid A or a portion thereof as described herein is used as a starting point for the rational design of a small molecule compound or compounds.

In other embodiments, the test compounds are structural analogs of a portion of MD-2 or TLR-4 that is involved in the binding of MD-2 to TLR4.

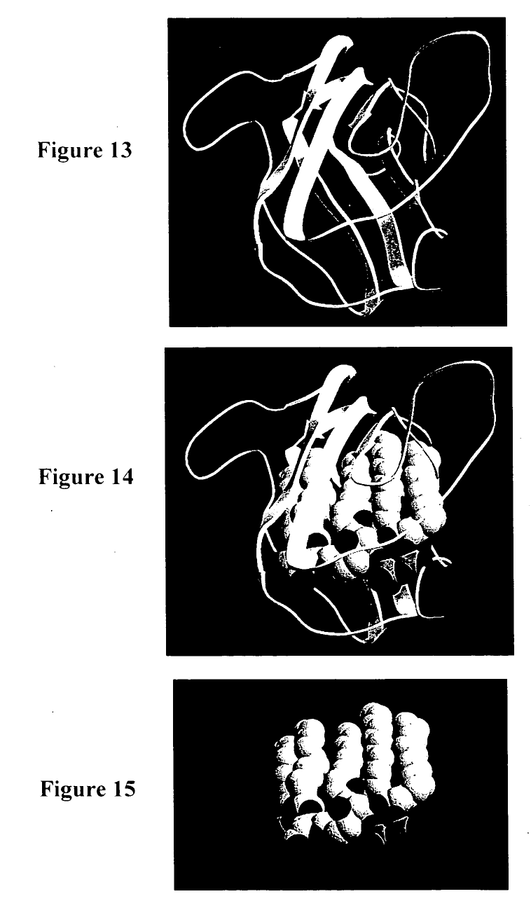

A computer-generated theoretical model of the structure of MD-2 has been created, see Gruber et al., J Biol Chem, 2004. 279(27): p. 28475-82, and is available at www.pdb.org, under Protein Data Base ID: 1T2Z. FIGS. 9 and 13 illustrate the structure of MD-2 from different viewpoints. MD-2 is described in Kato et al., Blood 96:362-364 (2000), and Shimazu et al., J. Exp. Med. 189:1777-1782 (1999). The sequence of human MD-2 is as follows (UniProt/Swiss-Prot|Q9Y6Y9|LY96_HUMAN Lymphocyte antigen 96 precursor; SEQ ID NO:2):

| | |
|---|---|
| MLPFLFFSTLFSSIFTEAQKQYWVCNSSDASISYTYCDKMQYPISIN | 50 |
| VNP | |
| CIELKGSKGLLHIFYIPRRDLKQLYFNLYITVNTMNLPKRKEVICRG | 100 |
| SDD | |
| DYSFCRALKGETVNTTISFSFKGIKFSKGKYKCVVEAISGSPEEMLF | 150 |
| CLE | |
| FVILHQPNSN | 160 |

Those resides important in binding LPS (Lysines 128 and 132 of SEQ ID NO:2) and in signalling (Cysteines 95 and 105) are highlighted in space-filling mode in FIG. 9.

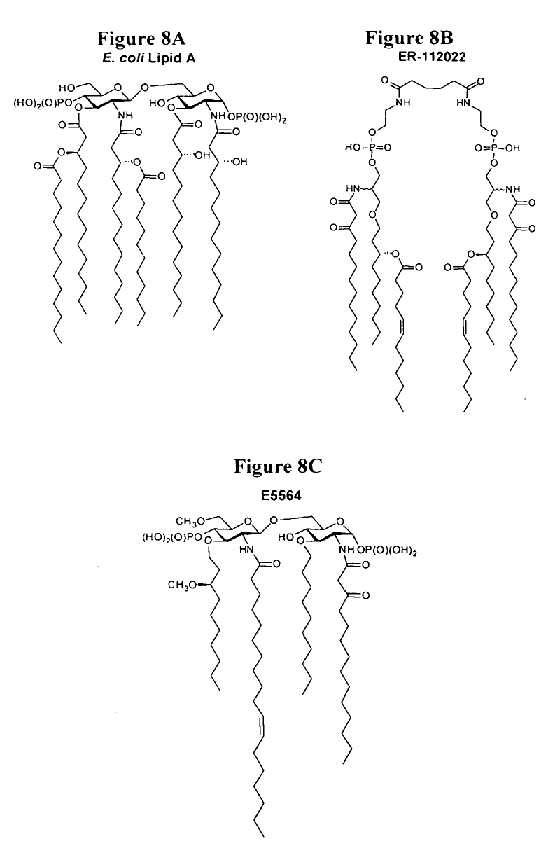

Bacterial lipopolysaccharides (LPS) typically consist of a hydrophobic domain known as lipid A (or endotoxin). Although there are numerous variants of lipid A, they are all characterized by a number of acyl moieties attached to a disaccharide-containing end. FIG. 8A is a schematic illustration of the chemical structure of lipid A as found in E. coli strains. FIGS. 8B and 8C illustrate the LPS agonist ER-112022 and antagonist E5564, respectively (both are from Eisai Research Institute in Andover, Mass.). FIGS. 10 and 15 are two views of a space-filling model of lipid A, generated using empirical data from a crystal in which LPS co-crystallized (available at www.pdb.org, under PDB ID:1QFF). In some embodiments, a portion of lipid A including the disaccharide-containing end is used as the starting point to generate analogs. In some embodiments, the methods include using computer modeling methods for rational drug design that are known in the art to evaluate and optimize structures of lipid A analogs for interaction with MD-2. FIGS. 11, 12, and 14 show a model of LPS lipid A bound in the hydrophobic pocket of MD-2.

A number of methods are known in the art for making structural analogs of a molecule. The following non-limiting examples are programs, including their user guides and manuals, suitable for generating, searching, and designing molecular structures: Concord (Tripos Associated, St. Louis, Mo.), 3-D Builder (Chemical Design Ltd., Oxford, U.K.), Catalyst (Bio-CAD Corp., Mountain View, Calif.), Daylight and DISCO (Abbott Laboratories, Abbott Park, Ill.); Ludi (Biosym Technologies Inc., San Diego, Calif.); Aladdin (Daylight Chemical Information Systems, Irvine Calif.; and ChemDBS-3D (Chemical Design Ltd., Oxford, U.K.). A database of chemical structures, e.g., a database available from Cambridge Crystallographic Data Centre (Cambridge, U.K.) and Chemical Abstracts Service (Columbus, Ohio) can be searched with the appropriate constraints using computer-based programs such as: MACCS-3D and ISIS/3D (Molecular Design Ltd., San Leandro, Calif.), ChemDBS-3D (Chemical Design Ltd., Oxford, U.K.), and Sybyl/3DB Unity (Tripos Associates, St. Louis, Mo.). Examples and reviews of pharmacophore discovery are also described in Milne et al. (1998) SAR QSAR Environ Res 9:23-38; Hong et al. (1997) J. Med. Chem. 40:920-936; Mason and Cheney (2000) Pac. Symp. Biocomput. 576-87; Ekins et al. (2000) Drug Metab Dispos 28:994-1002; Fradera et al. (2000) Proteins 40:623-636; and Schneider et al. (2000) J. Comput. Aided Mol. Des. 14:487-494.

Compounds that interfere with LPS binding to MD-2 or with MD-2 binding to TLR4 can be identified or designed by a method that includes using a representation of human MD-2 or a fragment thereof, or a complex of human MD-2 bound to LPS, e.g., bound to the lipid A portion of LPS, or a complex of MD-2 bound to TLR4, or a fragment of either one of these.

Various software programs allow for the graphical representation of a set of structural coordinates to obtain a representation of a complex of the human MD-2 bound to LPS, or a complex of MD-2 bound to TLR4. In general, such a representation should accurately reflect (relatively and/or absolutely) theoretical or actual structural coordinates, or information derived from structural coordinates, such as distances or angles between features. In some embodiments, the structural coordinates are derived empirically. For example, x-ray crystallography or NMR can be used to obtain structural coordinates of a complex of human MD-2 bound to LPS, e.g., bound to lipid A, or a complex of MD-2 bound to TLR4. Additional structural information can be obtained from spectral techniques (e.g., optical rotary dispersion (ORD), circular dichroism (CD)), homology modeling, and computational methods (e.g., computational methods that can include data from molecular mechanics, computational methods that include data from dynamics assays). In some embodiments, the structural coordinates are derived computationally by analogy to a structurally- or functionally-related protein, e.g., a related protein the structure of which has been determined by x-ray crystallography.

In some embodiments, the representation is a two-dimensional figure, such as a stereoscopic two-dimensional figure. In certain embodiments, the representation is an interactive two-dimensional display, such as an interactive stereoscopic two-dimensional display. An interactive two-dimensional display can be, for example, a computer display that can be rotated to show different faces of a polypeptide, a fragment of a polypeptide, a complex and/or a fragment of a complex. In some embodiments, the representation is a three-dimensional representation. As an example, a three-dimensional model can be a physical model of a molecular structure (e.g., a ball-and-stick model). As another example, a three dimensional representation can be a graphical representation of a molecular structure (e.g., a drawing or a figure presented on a computer display). A two-dimensional graphical representation (e.g., a drawing) can correspond to a three-dimensional representation when the two-dimensional representation reflects three-dimensional information, for example, through the use of perspective, shading, or the obstruction of features more distant from the viewer by features closer to the viewer. In some embodiments, a representation can be modeled at more than one level. As an example, when the three-dimensional representation includes a polypeptide, such as a complex of the human MD-2 bound to LPS, e.g., bound to the lipid A portion of LPS, or a complex of MD-2 bound to TLR4, the MD-2 and/or TLR4 polypeptide can be represented at one or more different levels of structure, such as primary (amino acid sequence), secondary (e.g., α-helices and β-sheets), tertiary (overall fold), and quaternary (oligomerization state) structure. A representation can include different levels of detail. For example, the representation can include the relative locations of secondary structural features of a protein without specifying the positions of atoms. A more detailed representation could, for example, include the positions of atoms.

In some embodiments, a representation can include information in addition to the structural coordinates of the atoms in a complex of the human MD-2 bound to LPS, or a complex of MD-2 bound to TLR4. For example, a representation can provide information regarding the shape of a solvent accessible surface, the van der Waals radii of the atoms of the model, and the van der Waals radius of a solvent (e.g., water). Other features that can be derived from a representation include, for example, electrostatic potential, the location of voids or pockets within a macromolecular structure, and the location of hydrogen bonds and salt bridges.

In some embodiments, the representation can be of an MD-2 polypeptide complexed with a compound that is known to bind to MD-2, e.g., a competitive agonist or antagonist of LPS, e.g., an analog of lipid A, e.g., an analog as described herein, e.g., E5564 or ER-112022. In some embodiments, the representation can be of a complex of MD-2 bound to TLR4, e.g., the extracellular portion of TLR4.

A candidate compound that interacts with the representation can be designed or identified by performing computer fitting analysis of the candidate compound with the representation. A compound that interacts with a polypeptide can interact transiently or stably with the polypeptide. The interaction can be mediated by any of the forces noted herein, including, for example, hydrogen bonding, electrostatic forces, hydrophobic interactions, and van der Waals interactions.

X-ray diffraction data can be used to construct an electron density map of a complex of MD-2 bound to LPS, e.g., bound to the lipid A portion of LPS, or of a complex of MD-2 bound to TLR4, and the electron density map can be used to derive a representation (e.g., a two dimensional representation, a three dimensional representation) of MD-2 bound to LPS or of MD-2 bound to TLR4. Creation of an electron density map typically involves using information regarding the phase of the X-ray scatter. Phase information can be extracted, for example, either from the diffraction data or from supplementing diffraction experiments to complete the construction of the electron density map. Methods for calculating phase from X-ray diffraction data include, for example, multiwavelength anomalous dispersion (MAD), multiple isomorphous replacement (MIR), multiple isomorphous replacement with anomalous scattering (MIRAS), single isomorphous replacement with anomalous scattering (SIRAS), reciprocal space solvent flattening, molecular replacement, or any combination thereof. Upon determination of the phase, an electron density map can be constructed. The electron density map can be used to derive a representation of the complex or a fragment thereof by aligning a three-dimensional model of a previously known polypeptide or a previously known complex (e.g., a complex containing a polypeptide bound to a ligand) with the electron density map. For example, a hypothetically-derived electron density map corresponding to a complex of MD-2 and a test compound can be aligned with an empirically or computationally derived electron density map corresponding to MD-2/lipid A complex, e.g., as described herein.

The alignment process results in a comparative model that shows the degree to which the calculated electron density map varies from the model of the previously known polypeptide or the previously known complex. The comparative model is then refined over one or more cycles (e.g., two cycles, three cycles, four cycles, five cycles, six cycles, seven cycles, eight cycles, nine cycles, 10 cycles) to generate a better fit with the electron density map. A software program such as CNS (Brunger et al., Acta Crystallogr. D54:905-921, 1998) can be used to refine the model. The quality of fit in the comparative model can be measured by, for example, an R_{work} or R_{free} value. A smaller value of R_{work} or R_{free} generally indicates a better fit. Misalignments in the comparative model can be adjusted to provide a modified comparative model and a lower R_{work} or R_{free} value. The adjustments can be based on information (e.g., structural or sequence information) relating to MD-2, TLR4, lipid A, and/or the test compound. As an example, in embodiments in which a model of a previously known complex of an MD-2 or TLR4 polypeptide bound to a ligand is used, an adjustment can include replacing the ligand in the previously known complex with a test compound. When adjustments to the modified comparative model satisfy a best fit to the electron density map, the resulting model is that which is determined to best describe the complex. Methods of such processes are disclosed, for example, in Carter and Sweet, eds., “Macromolecular Crystallography” in Methods in Enzymology, Vol. 277, Part B, (New York, Academic Press, 1997), and articles therein, e.g., Jones and Kjeldgaard, “Electron-Density Map Interpretation,” p. 173, and Kleywegt and Jones, “Model Building and Refinement Practice,” p. 208.

A machine, such as a computer, can be programmed in memory with the structural coordinates of a complex of MD-2 bound to lipid A, or a complex of MD-2 bound to TLR4, together with a program capable of generating a graphical representation of the structural coordinates on a display connected to the machine. Alternatively or additionally, a software system can be designed and/or utilized to accept and store the structural coordinates. The software system can be capable of generating a graphical representation of the structural coordinates. The software system can also be capable of accessing external databases to identify compounds (e.g., small molecules) with similar structural features to, e.g., lipid A, TLR4, or MD-2, and/or to identify one or more candidate compounds with characteristics that may render the candidate compound(s) likely to interact with MD-2, e.g., human MD-2, or TLR4, e.g., in such a way as to interfere with binding of TLR4 and MD-2 or MD-2 and LPS.

A machine having a memory containing structure data or a software system containing such data can aid in the rational design or selection of LPS agonists and/or antagonists. For example, such a machine or software system can aid in the evaluation of the ability of a compound to associate with MD-2, e.g., soluble MD-2 or MD-2 bound to TLR4, or can aid in the modeling of compounds related by structural homology to lipid A. As used herein, an LPS agonist refers to a compound that mimics or enhances at least one activity of LPS, and an LPS antagonist refers to a compound that inhibits at least one activity, or has an opposite activity, of LPS. An activity of LPS can be, e.g., activation of TLR4 signalling.

The machine can produce a representation (e.g., a two dimensional representation, a three dimensional representation) of a complex of MD-2 bound to LPS or a fragment thereof, e.g., lipid A. A software system, for example, can cause the machine to produce such information. The machine can include a machine-readable data storage medium including a data storage material encoded with machine-readable data. The machine-readable data can include structural coordinates of atoms of a complex of MD-2 bound to LPS or a fragment thereof, e.g., lipid A. Machine-readable storage media (e.g., data storage material) include, for example, conventional computer hard drives, floppy disks, DAT tape, CD-ROM, DVD, and other magnetic, magneto-optical, optical, and other media which may be adapted for use with a machine (e.g., a computer). The machine can also have a working memory for storing instructions for processing the machine-readable data, as well as a central processing unit (CPU) coupled to the working memory and to the machine-readable data storage medium for the purpose of processing the machine-readable data into the desired three-dimensional representation. A display can be connected to the CPU so that the three-dimensional representation can be visualized by the user. Accordingly, when used with a machine programmed with instructions for using the data (e.g., a computer loaded with one or more programs of the sort described herein) the machine is capable of displaying a graphical representation (e.g., a two dimensional graphical representation, a three-dimensional graphical representation) of any of the polypeptides, polypeptide fragments, complexes, or complex fragments described herein.

A display (e.g., a computer display) can show a representation of a complex of MD-2 bound to LPS or a fragment thereof, e.g., lipid A, or a complex of MD-2 bound to TLR4. The user can inspect the representation and, using information gained from the representation, generate a model of a complex or fragment thereof that includes a compound other than those previously present, e.g., other than LPS or lipid A, or other than MD-2. The model can be generated, for example, by altering a previously existing representation of MD-2 bound to LPS or a fragment thereof, e.g., lipid A, or a previously existing representation of MD-2 bound to TLR4. Optionally, the user can superimpose a three-dimensional model of a test compound on, e.g., the representation of MD-2 bound to LPS or a fragment thereof, e.g., lipid A. The compound can be an LPS agonist (e.g., a candidate agonist) or antagonist (e.g., a candidate antagonist). In some embodiments, the compound can be a known compound or fragment of a compound. In certain embodiments, the compound can be a previously unknown compound, or a fragment of a previously unknown compound.

It can be desirable for the compound to have a shape that complements the shape of the active site, e.g., of the hydrophobic pocket of MD-2, e.g., and interacts with one or both, preferably both, of Lys 128 and Lys 132. There can be a preferred distance, or range of distances, between atoms of the compound and atoms of the MD-2 polypeptide, e.g., Lys 128 and Lys 132. Distances longer than a preferred distance may be associated with a weak interaction between the compound and active site. Distances shorter than a preferred distance may be associated with repulsive forces that can weaken the interaction between the compound and the polypeptide. A steric clash can occur when distances between atoms are too short. A steric clash occurs when the locations of two atoms are unreasonably close together, for example, when two atoms are separated by a distance less than the sum of their van der Waals radii. If a steric clash exists, the user can adjust the position of the compound relative to the MD-2 polypeptide (e.g., a rigid body translation or rotation of the compound), until the steric clash is relieved. The user can adjust the conformation of the compound or of the MD-2 polypeptide in the vicinity of the compound to relieve a steric clash. Steric clashes can also be removed by altering the structure of the compound, for example, by changing a “bulky group,” such as an aromatic ring, to a smaller group, such as to a methyl or hydroxyl group, or by changing a rigid group to a flexible group that can accommodate a conformation that does not produce a steric clash. Electrostatic forces can also influence an interaction between a compound and a ligand-binding domain. For example, electrostatic properties can be associated with repulsive forces that can weaken the interaction between the compound and the MD-2 or TLR4 polypeptide. Electrostatic repulsion can be relieved by altering the charge of the compound, e.g., by replacing a positively charged group with a neutral group.

Forces that influence binding strength between a test compound and MD-2 or TLR4 can be evaluated in the polypeptide/compound model. These can include, for example, hydrogen bonding, electrostatic forces, hydrophobic interactions, van der Waals interactions, dipole-dipole interactions, π-stacking forces, and cation-π interactions. The user can evaluate these forces visually, for example by noting a hydrogen bond donor/acceptor pair arranged with a distance and angle suitable for a hydrogen bond. Based on the evaluation, the user can alter the model to find a more favorable interaction between the MD-2 or TLR4 polypeptide and the compound. Altering the model can include changing the three-dimensional structure of the polypeptide without altering its chemical structure, for example by altering the conformation of amino acid side chains or backbone dihedral angles. Altering the model can include altering the position or conformation of the compound, as described above. Altering the model can also include altering the chemical structure of the compound, for example by substituting, adding, or removing groups. For example, if a hydrogen bond donor on the MD-2 or TLR4 polypeptide is located near a hydrogen bond donor on the compound, the user can replace the hydrogen bond donor on the compound with a hydrogen bond acceptor.

The relative locations of a compound and the MD-2 or TLR4 polypeptide, or their conformations, can be adjusted to find an optimized binding geometry for a particular compound to the polypeptide. An optimized binding geometry is characterized by, for example, favorable hydrogen bond distances and angles, maximal electrostatic attractions, minimal electrostatic repulsions, the sequestration of hydrophobic moieties away from an aqueous environment, and the absence of steric clashes. The optimized geometry can have the lowest calculated energy of a family of possible geometries for an MD-2 polypeptide/compound complex. An optimized geometry can be determined, for example, through molecular mechanics or molecular dynamics calculations.

A series of representations of complexes of MD-2 and/or TLR4 with different bound compounds can be generated. A score can be calculated for each representation. The score can describe, for example, an expected strength of interaction between the polypeptide and the compound. The score can reflect one of the factors described above that influence binding strength. The score can be an aggregate score that reflects more than one of the factors. The different compounds can be ranked according to their scores.

Steps in the design of the compound can be carried out in an automated fashion by a machine. For example, a representation of MD-2 and/or TLR4 can be programmed in the machine, along with representations of candidate compounds. The machine can find an optimized binding geometry for each of the candidate compounds to the active site, and calculate a score to determine which of the compounds in the series is likely to interact most strongly with MD-2 and/or TLR4.

A software system can be designed and/or implemented to facilitate these steps. Software systems (e.g., computer programs) used to generate representations or perform the fitting analyses include, for example: MCSS, Ludi, QUANTA, Insight II, Cerius2, CHarMM, and Modeler from Accelrys, Inc. (San Diego, Calif.); SYBYL, Unity, FleXX, and LEAPFROG from TRIPOS, Inc. (St. Louis, Mo.); AUTODOCK (Scripps Research Institute, La Jolla, Calif.); GRID (Oxford University, Oxford, UK); DOCK (University of California, San Francisco, Calif.); and Flo+ and Flo99 (Thistlesoft, Morris Township, N.J.). Other useful programs include ROCS, ZAP, FRED, Vida, and Szybki from Openeye Scientific Software (Santa Fe, N. Mex.); Maestro, Macromodel, and Glide from Schrodinger, LLC (Portland, Oreg.); MOE (Chemical Computing Group, Montreal, Quebec), Allegrow (Boston De Novo, Boston, Mass.), and GOLD (Jones et al., J. Mol. Biol. 245:43-53, 1995). The structural coordinates can also be used to visualize the three-dimensional structure of an ERalpha polypeptide using MOLSCRIPT, RASTER3D, or PYMOLE (Kraulis, J. Appl. Crystallogr. 24: 946-950, 1991; Bacon and Anderson, J. Mol. Graph. 6: 219-220, 1998; DeLano, The PyMOL Molecular Graphics System (2002) DeLano Scientific, San Carlos, Calif.).

The compound can, for example, be selected by screening an appropriate database, can be designed de novo by analyzing the steric configurations and charge potentials of unbound MD-2 in conjunction with the appropriate software systems, and/or can be designed using characteristics of known ligands of progesterone receptors or other hormone receptors. The method can be used to design or select LPS agonists or antagonists. A software system can be designed and/or implemented to facilitate database searching, and/or compound selection and design.

Once a compound has been designed or identified, it can be obtained or synthesized and further evaluated for its effect on LPS binding to MD2, MD-2 binding to TLR4, and/or LPS activity, e.g., using a method described herein. A method for evaluating the compound can include an activity assay performed in vitro or in vivo. An activity assay can be a cell-based assay, for example. A crystal containing MD-2 and/or TLR4 bound to the identified compound can be grown and the structure determined by X-ray crystallography and/or NMR. A second compound can be designed or identified based on the interaction of the first compound with MD-2 or TLR4.

Various molecular analysis and rational drug design techniques are further disclosed in, for example, U.S. Pat. Nos. 5,834,228, 5,939,528 and 5,856,116, as well as in PCT Application No. PCT/US98/16879, published as WO 99/09148.

Test compounds identified as “hits” (e.g., test compounds that interfere with LPS/MD-2 or TLR4/MD-2 binding) in a first screen can be selected and systematically altered, e.g., using rational design, to optimize binding affinity, avidity, specificity, or other parameter. Such optimized compounds can also be screened using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hits, and screening the second library using the methods described herein.

The test compounds can be, e.g., natural products or members of a combinatorial chemistry library. A set of diverse molecules can be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

As used herein, “small molecules” refers to small organic or inorganic molecules of molecular weight below about 5,000 Daltons.

Libraries screened using the methods of the present invention can include a variety of types of test compounds. A given library can include a set of structurally related or unrelated test compounds. In some embodiments, the test compounds are peptide or peptidomimetic molecules. In some embodiments, the test compounds are nucleic acids, e.g. aptamers. In some embodiments, the test compounds include one or more saccharide or polysaccharide moieties. In some embodiments, the test compounds are small molecules.

Pharmaceutical Compositions

The methods described herein include the manufacture and use of pharmaceutical compositions that include, e.g., as active ingredients, compounds identified by a method described herein, e.g., improved therapeutic versions of lipid A, or a TLR4:Fc. Also included herein are the pharmaceutical compositions themselves.

In some embodiments, the pharmaceutical compositions include a TLR4 extracellular domain fused to an FC region from an IgG type antibody, e.g., a TLR4:Fc construct, e.g., as described in U.S. Provisional Patent Application Ser. No. 60/598,774, filed on Aug. 4, 2004, the entire contents of which are incorporated herein by reference. Where the composition includes a TLR4:Fc, the composition and dosing can be formulated similarly to etanercept (Enbrel™, Wyeth Pharmaceuticals), a dimeric fusion protein consisting of the extracellular ligand-binding portion of the human 75 kilodalton (p75) tumor necrosis factor receptor (TNFR) linked to the Fc portion of human IgG1. For example, the TLR4:Fc can be provided in a solution for subcutaneous injection, formulated at pH 6.3±0.2, with 10 mg/mL sucrose, 5.8 mg/mL sodium chloride, 5.3 mg/mL L-arginine hydrochloride, 2.6 mg/mL sodium phosphate, monobasic, monohydrate, and 0.9 mg/mL sodium phosphate, dibasic, anhydrous. Alternatively, the TLR4:Fc can be provided as a sterile, white, preservative-free, lyophilized powder.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with their intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Methods of Prevention and Treatment

The methods described herein include methods for the prevention and treatment of disorders associated with TLR4 signalling, e.g., sterile inflammation (e.g., rheumatoid arthritis, psoriasis, or Crohn's disease) or infection with gram negative bacteria. In some embodiments, the disorder is sepsis or septic shock. Generally, the methods include administering a therapeutically effective amount of a therapeutic composition, e.g., a composition including a TLR4:Fc, or an analog of lipid A, as described herein, to a subject who is in need of, or who has been determined to be in need of, such treatment.

As used in this context, to “treat” means to ameliorate at least one symptom of the disorder.

Septic shock is usually preceded by sepsis, which is marked by shaking, chills, fever, weakness, confusion, nausea, vomiting, and diarrhea. Early signs of septic shock include confusion and decreased consciousness; shaking chills; a rapid rise in temperature; warm, flushed skin; a rapid, pounding pulse; excessively rapid breathing; and blood pressure that rises and falls. As the shock progresses the extremities become cool, pale, and bluish over time, and fever may give way to lower than normal temperatures. In some embodiments, the methods include administering a compound described herein, e.g., a TLR4:Fc, to a subject who is exhibiting one or more symptoms of sepsis, to prevent the development of septic shock.

Other symptoms of shock include rapid heartbeat, shallow, rapid respiration, decreased urination, and reddish patches in the skin. In some cases, septic shock progresses to “adult respiratory distress syndrome (ARDS),” in which fluid collects in the lungs, and respiration becomes very shallow and labored. ARDS may lead to ventilatory collapse, in which the subject can no longer breathe adequately without assistance.

Symptoms of sterile inflammation, e.g., rheumatoid arthritis, include those listed in the American College of Rheumatology (ACR) response criteria, which include changes in number of swollen joints, tender joints, physician global assessment of disease, patient global assessment of disease, patient assessment of pain, C-reactive protein (CRP), erythrocyte sedimentation rate (ESR), and health assessment questionnaire (HAQ) score. In some embodiments, treating results in at least an ACR₂₀ response, in which the subject has a 20% reduction in the number of swollen and tender joints, and a reduction of 20% in three of the following five indices: physician global assessment of disease, patient global assessment of disease, pain, CRP/ESR and HAQ.

In some embodiments, the methods include preventive methods, e.g., methods including administering a therapeutically effective amount of a composition described herein to a subject who is at risk of having a gram negative infection, e.g., subjects at the highest risk for developing sepsis, such as those with penetrating trauma to the abdomen or large bowel incarceration. In some embodiments, the methods further include the administration of an appropriate antibiotic, as known in the art.

Dosage, toxicity, and therapeutic efficacy of the compounds can be determined, e.g., by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods described herein, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

In some embodiments, the methods include administering a compound described herein with an antibiotic, e.g., as known in the art, or other treatments for shock, e.g., fluid given intravenously to increase the blood pressure, pharmaceuticals to increase blood flow to the brain, heart, and other organs, or extra oxygen. If the lungs fail, the person may need a mechanical ventilator to help breathing.

Blood Purification Therapy

The methods of treating sepsis described herein can include the use of blood purification methods. These methods typically include temporarily removing blood from a subject, treating the blood with TLR4, e.g., TLR4:Fc, to remove soluble MD-2 (sMD-2), and returning the blood to the subject. General methods for performing such purifications (sometimes referred to as “apheresis”) are known in the art, and typically involve passing the blood over a column or other device to extract a selected impurity, see, e.g., U.S. Pat. No. 6,569,112 (Strahilevitz); Asahi et al., Therapeutic Apheresis 7(1):74-77(5), 2003; Hout et al., ASAIO J., 46(6):702-206, 2000; Matsuo et al., Therapeutic Apheresis and Dialysis 8(3):194, 2004. These methods that are known in the art can be adapted for use in the present method. For example, a column including the TLR4, e.g., as TLR4:Fc can be constructed using methods known in the art, and the blood can be passed through it, removing a substantial amount of the sMD-2 present in the blood. Alternatively, collectible beads, e.g., magnetic beads, can be coated with TLR4:Fc, and the blood can be mixed with the beads, and the beads then extracted to removed the sMD-2. In some embodiments, the blood is separated into its components before being passed over the column or contacted with the beads. In some embodiments, the methods can be used to remove LPS from the blood, by using a column or other collectible substrate with covalently linked TLR4:Fc/MD-2, which will pull LPS out of the blood.

Methods of Diagnosis

Included herein are methods for diagnosing infection with gram negative bacteria. The methods include obtaining a sample from a subject, and evaluating the presence and/or level of soluble MD-2 (sMD-2) in the sample, and comparing the presence and/or level with one or more references, e.g., a control reference that represents a normal level of sMD-2, e.g., a level in an unaffected subject, and/or a disease reference that represents a level of sMD-2, associated with infection with gram negative bacteria, e.g., a level in a subject having an infection with gram negative bacteria. Suitable reference values can include those described herein, e.g., those shown in Example 7, e.g., 1.5 nM, such that levels statistically significantly above the reference, e.g., 1.5 nM, indicate that the subject has a gram negative bacterial infection. The presence and/or level of a protein can be evaluated using methods described herein, or other methods known in the art.

In some embodiments, the presence and/or level of sMD-2 is comparable to the presence and/or level sMD-2 in the disease reference, and if the subject also has one or more symptoms associated with a gram negative bacterial infection, then the subject has a gram negative bacterial infection. In some embodiments, the subject has no overt signs or symptoms of a gram negative bacterial infection, but the presence and/or level of sMD-2 is comparable to the presence and/or level of sMD-2 in the disease reference, then the subject has a gram negative bacterial infection. In some embodiments, the sample includes a biological fluid, e.g., blood, semen, urine, and/or cerebrospinal fluid. In some embodiments, once it has been determined that a person has a gram negative bacterial infection, then a treatment, e.g., as known in the art or as described herein, can be administered.

Also included herein are methods for detecting endotoxin (LPS) in biological or other samples, e.g., fluids such as blood or water. The methods include obtaining a sample, and evaluating the presence and/or level of LPS in the sample using an assay described herein, e.g., an assay that detects the presence and/or level of LPS in the sample by detecting the presence of a compound that competes for binding MD-2, e.g., recombinant MD-2 (soluble or bound to a surface, e.g., a slide or capillary membrane, e.g., as in a sandwich ELISA) with LPS, e.g., a known quantity of LPS, e.g., labeled LPS. In some embodiments, the methods include comparing the presence and/or level with one or more references, e.g., a control reference that represents a preselected level of LPS, e.g., a level above which the fluid is unsafe to use. These methods can be used in place of, or in addition to, Limulus amoebocyte lysate assays, which have limited use in blood (see, e.g., Hurley, Clinical Microbiology Reviews, 8(2):268-292 (1995). In some embodiments, the sample is from a subject, and the presence of LPS in the sample indicates that the subject has a gram-negative infection. These methods have the advantage that LPS from a wide variety of bacterial sources will be detected, as opposed to methods such as PCR-based methods that may only detect one or a subset of bacteria. The methods can be used, e.g., to detect endotoxin in donated blood before transfusion, in liquids to be used for cell culture, or in drinking water. In some embodiments, the assay is a simple yes/no assay, and the results indicate that endotoxin is present in an unacceptable level. In some embodiments, the assay indicates what level of endotoxin is present.

The invention also includes kits for detecting the presence of endotoxin using a method described herein. The kits can include MD-2, e.g., MD-2 bound to a solid surface such as a slide; directly or indirectly labeled LPS (or an MD-2 binding portion thereof, e.g., lipid A), e.g., a known quantity of LPS; reagents for detecting binding of the LPS to the MD-2, e.g., avidin-HRP, if the LPS is biotinylated; and a reference, e.g., a reference that represents a selected level of endotoxin, e.g., a level of endotoxin above which a tested sample is not usable, or a number of references, e.g., to allow quantification of the level of endotoxin present in the sample. The kit can include directions for practicing a method described herein to detect the presence or determine the level of endotoxin in the sample.

The invention is further described in the following examples, which do not limit the scope of the invention described in the claims.

### EXAMPLES

Cells, Constructs and Reagents

Unless otherwise stated, reagents were obtained from Sigma Chemicals, Inc. (St. Louis. Mo.). The TLR4^{YFP} cell line used in this study was previously described (11). Cell lines were maintained in “complete medium” (5% FCS in DMEM plus 10 g of ciprofloxacin per ml) in a humidified 5% CO₂ atmosphere. Cells stably secreting the TLR:Fc chimeric constructs were generated by retroviral transduction of HEK293 cells (ATCC CRL-1573; gift of Jesse Chow, Eisai Research Institute, Andover, Mass.) and were maintained in protein free medium (293PF, Invitrogen, Carlsbad, Calif.). The TLR fusion proteins consisted of the entire extracellular domain of either human TLR2 (amino acids 1-587) or hTLR4 (amino acids 1-632) fused in frame with the C-terminal 233 amino acid Fc portion of mouse IgG₂ₐ, modified by the addition of the linker sequence GAAGG (SEQ ID NO:3). The cDNA for human MD-2 was PCR cloned from the original pEF-Bos-MD-2^{FLAG/6×His} provided by Dr. K. Miyake (University of Tokyo, Tokyo, Japan), and subcloned into the retroviral vector pCLCX4 (23). The resulting construct encodes for an N-terminally tagged FLAG and a C-terminally 6×His tagged human MD-2. Packaging of the virus and transduction of HEK293 cells was performed as described (23). Antibodies used in this study included: rabbit polyclonal anti-GFP antibody from Molecular Probes (Eugene, Oreg.); HRP-conjugated anti-GFP rabbit antiserum from Abcam (Cambridge, Mass.); mouse monoclonal anti-GFP from Clontech (Bedford, Mass.); mouse monoclonal anti TLR4, clone HTA125 from Dr. K. Miyake; anti 6×His monoclonal antibody from Novagen-EMD Biosciences (La Jolla, Calif.); rabbit polyclonal HRP-conjugated anti biotin from New England Biolabs (Beverly, Mass.). The lipid A antagonist E5564 (24) and the agonist ER112022 were provided by the Eisai Research Institute (Andover, Mass.). The baculovirus encoding for a C-terminally 6×His tagged human MD-2 was provided by Dr. S. Viriyakosol (University of California San Diego), and expanded in Sf9 insect cells. MD-2 was then purified as described in (9). Soluble recombinant CD14 and LBP were gifts from Amgen (Thousand Oaks, Calif.).

### Example 1

### MD-2 Binds to Living S. typhimurium

The existence of soluble MD-2 was first demonstrated by in vitro studies of overexpressed recombinant MD-2 in HEK293 supernatants (8). As described herein, human serum from apparently healthy individuals contains soluble MD-2 (see Example 7, below). Since MD-2 can readily interact with endotoxin, it was hypothesized that it may also be able to interact with intact Gram-negative bacteria, in a way that is similar to soluble CD14 or LBP (7, 8). To test this hypothesis, recombinant MD-2 was expressed as a 6×His-tagged molecule in baculovirus and the recombinant protein was purified from supernatants. These preparations of MD-2 consisted primarily of oligomeric MD-2, but contained approximately 50% monomeric MD-2. Salmonella typhimurium (serotype SB3201) was grown overnight in liquid LB-broth cultures, and harvested from the broth cultures in stationary phase. Cells were washed twice in PBS-1% BSA to remove possible MD-2 ligands in the bacterial culture medium and were resuspended in PBS-BSA, in the presence or absence of recombinant purified 6×His tagged MD-2 at a concentration of 0.1 μg protein per 1.0×10⁸ bacteria. After 30 minutes incubation at room temperature, the cells were washed twice in PBS-BSA and 1 μg of anti-6×His monoclonal antibody was added to 100 μl of resuspended bacterial pellet and the cells incubated on ice for an additional 30 minutes. After two washings, an Alexa488 conjugated anti-mouse antibody (1:200, Molecular Probes, Eugene, Calif.) was added to detect the anti-6×His antibody. Cells were washed once more in PBS and were subjected to FACS analysis at 488 nm. In parallel experiments, bacteria were washed after the addition of MD-2, resuspended in reducing SDS-loading buffer and western blotted for MD-2 using an anti-6×His monoclonal antibody (Qiagen).

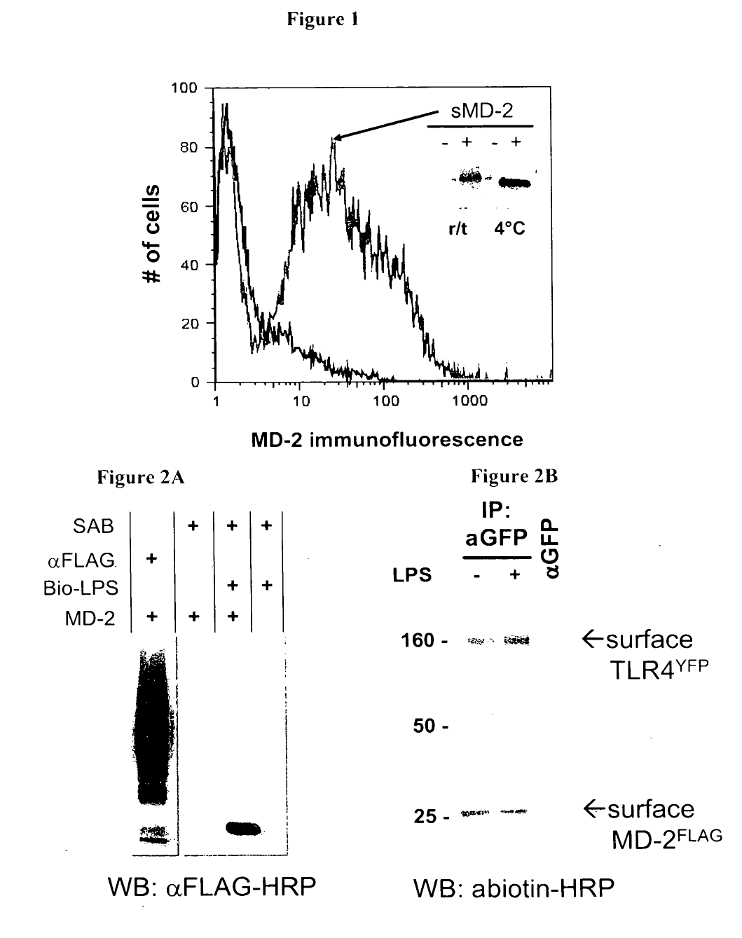

As shown in FIG. 1, the FACS profile shows an intense shift of fluorescence in the sample incubated with MD-2. The anti-6×His monoclonal antibody, although used at a tenfold concentration, gave negligible binding under these conditions.

The bacterial cells (incubated in the presence or absence of MD-2) were then subjected to SDS-PAGE and Western blot analysis with an anti-6×His monoclonal antibody (inset to FIG. 1) to detect bound MD-2. The protocol used for cell lysis and immunoprecipitation was described in detail in (4), which is incorporated herein by reference. Briefly, cells were grown in an adhesive monolayer in 10 cm dishes (˜7-8×10⁶ cells) and lysed by adding 1 ml of ice cold lysis buffer (20 mM Tris, pH 8, 137 mM NaCl, 1% Triton X-100, 2 mM EDTA, 10% Glycerol, and freshly added protease inhibitors {PMSF (1 nM), Leupeptin and Aprotinin (10 μg/ml)} to the cell monolayer. Lysates were subjected to centrifugation at 10000×g×5 minutes and the supernatants were incubated with 20 μl of packed protein A Sepharose™ resin (PAS, Amersham-Biosciences, Piscataway, N.J.) and 2 μg of the indicated antibody for either one hour or overnight at 4° C. Captured immunocomplexes were extensively washed in lysis buffer minus the protease inhibitors, and subjected to SDS-PAGE under reducing (0.1 M DTT in the loading buffer) or non reducing conditions, as indicated. When biotin-LPS was used to capture LPS-interacting proteins, 20 μl of packed streptavidin beads (SAB) were used instead of PAS, and biotin-LPS was used at a final concentration of either 1 or 0.5 μg/ml as indicated in the figure legends. When the Fc fusion constructs were precipitated, 20 μl of packed PAS beads were used without additional antibodies. Precast 4-16% polyacrylamide gels were purchased from VWR (Gradipore Inc., Frenchs Forest, Australia). The resolved proteins were electroblotted onto nitrocellulose membranes (Hybond C, Amersham, Piscataway, N.J.) that were blocked in non-fat powdered milk solubilized in PBS plus 0.1% Tween™ 20 (PBS-T).

The membranes were probed with 1 μg of the indicated HRP-conjugated antibody/ml in PBS-T for 30 minutes at room temperature. When a secondary HRP-conjugated antibody was required for detection, Bio-Rad anti-mouse or anti rabbit-antisera were used at a 1:5,000 dilution in PBS-T for an additional 15 minutes. After each step, membranes were washed in PBS-T for 10 minutes, and finally subjected to Enhanced Chemiluminescence (ECL) per manufacturer instructions (Amersham).

To quantify the 6×His tagged monomeric MD-2, a comparative Western blot analysis of purified MD-2 versus titrated amounts of a 30 kDa 6×His tagged protein standard (Qiagen, Valencia, Calif.) was performed. The concentration of the MD-2 stocks was ˜1 μM. Since the preparation of baculoviral MD-2 consisted of approximately 60% monomeric, 30% dimeric and 10% multimeric MD-2, the concentration of monomeric MD-2 was ˜0.6 μM.

These results demonstrate that soluble MD-2 binds to living S. typhimurium.

### Example 2

### Only Monomeric MD-2 Participates in TLR4 Activation

Multimeric forms of MD-2 can be observed when the molecule is overexpressed by 293 cells (FIG. 2A), although it is unknown if MD-2 multimerizes in vivo. It was hypothesized previously that multimerization of MD-2 might be responsible for the aggregation of TLR4, since the triggering of TLR4 can be efficiently achieved by antibody crosslinking of either TLR4 (11) or TLR4 bound MD-2 (26).

Our initial impression of MD-2 was distorted by the fact that the anti-FLAG mAb (M2) recognizes recombinant polymeric, but poorly recognizes monomeric soluble MD-2 when it is engineered with a FLAG epitope immediately downstream from the 6×His tag at the C-terminus (16, 17). Reengineering the MD-2 molecule with the FLAG epitope at the N-terminus allowed the production of a protein whose monomeric form was readily recognized by anti-FLAG antibody. The newly engineered MD-2 was subjected to the biotin-LPS pull down.

Protein binding to LPS was studied as described (4). Briefly, the polysaccharide of E. coli 's LPS (0111:B4) was labeled using hydrazide-biotin as per the manufacturer instructions (Pierce). Biotinylated LPS was gel filtered in HANK's balanced solution to remove free biotin, tested for activity and stored at 4° C. The assay is designed to detect the interaction of epitope-tagged recombinant proteins with LPS. To detect LPS binding to soluble proteins, biotin-LPS (0.5 or 1 μg/ml) and SAB (20 μl packed resin/point) were mixed with culture supernatants from transfected cells that secrete the candidate proteins for one hour at 37° C. or overnight at 4° C. In order to detect LPS binding to proteins that are expressed on the surface of cells, biotin-LPS was added to 5 ml of medium covering monolayers of growing cells at a final concentration of 1 μg/ml, and treated for 1 hour in a 37° C. incubator in a 5% CO₂ humidified atmosphere. Cells were lysed as described (4), and lysates were subjected to centrifugation at 10,000×g×5 min to remove insoluble material. LPS-interacting proteins were captured using 20 μl of packed SAB/sample directly added to the post nuclear whole cell lysates. Beads were then extensively washed in lysis buffer and proteins were eluted from the beads by boiling in SDS sample buffer. LPS-precipitated proteins were resolved by SDS-PAGE and Western blotted using antibodies to their epitope tags (e.g., anti-GFP to detect TLR4^{YFP}, anti mouse to detect TLR4:Fc, anti-FLAG to detect MD-2^{FLAG} or TLR4^{FLAG}).

As shown in FIG. 2A, third lane, only the mature form of the monomeric MD-2 was precipitated.

In addition to the important role of monomeric MD-2 in binding LPS, this form of MD-2 was found to be the only form that interacts with TLR4 on the cell surface. HEK/TLR4 cells expressing the YFP tagged TLR4 and MD-2 were left untreated or were treated with 1 μg of LPS/ml for one hour followed by surface labeling using a non-membrane permeable biotinylation reagent (NHS-biotin). Cells were immunoprecipitated with anti-TLR4 mAb carried out as described in Example 1, followed by Western blotting with an anti-biotin mAb (FIG. 2B). To detect surface proteins, cells were surface biotinylated using 10 μg of the membrane impermeable compound NHS-biotin/ml per the manufacturer instructions (Pierce). Biotinylated proteins were detected in western blot by using an HRP-conjugated polyclonal anti-biotin antiserum (New England Biolabs) diluted 1:000 in PBS-T.

The addition of LPS prior to immunoprecipitation did not alter the aggregation status of TLR4 associated MD-2, ruling out the possibility that covalent multimerization of MD-2 is catalyzed by endotoxin or is in some way related to TLR4 aggregation (FIG. 2B, lane 2).

As only monomeric MD-2 binds LPS, and only monomeric MD-2 binds TLR4, these results demonstrate that only monomeric MD-2 participates in TLR4 activation by endotoxin. Therefore, the monomeric form is the biologically relevant form.

### Example 3

### Recognition of LPS by TLR4 and MD-2 does not Require Additional Cellular Components

An increasing amount of evidence is accumulating in the literature on the role of ancillary proteins in the LPS receptor complex, and in particular on the role of lipid rafts associated receptor in LPS signaling (27, 28). To establish whether the LPS recognition event by MD-2/TLR4 requires additional cellular co-factors, the LPS binding assay to TLR4 was performed in the soluble phase with purified receptor components. Supernatants from cells stably expressing a recombinant soluble TLR4 extracellular domain (TLR4:Fc) and supernatants from MD-2 expressing cells were tested for the ability of binding biotin-LPS.

Briefly, conditioned media from MD-2 and TLR4:Fc expressing cells were mixed in equal amounts (lanes 3-5) and proteins were captured with streptavidin beads in the presence (lanes 1, 3) or absence (lane 2) of biotinylated LPS (1 μg/ml). Samples were then subjected to biotin-LPS precipitation (lanes 1, 3) or protein A precipitation (lanes 4-6). After one hour incubation at room temperature, beads were washed in lysis buffer, and the captured proteins were eluted from the beads by addition of reducing SDS sample buffer. The eluted proteins were separated on a 4-16% polyacrylamide gel, blotted on a nitrocellulose membrane, blocked, and probed with HRP-labeled anti-mouse polyclonal Ab (for the TLR4:Fc chimera, upper portion of the membrane) or an anti-FLAG mAb (for MD-2^{FLAG}, bottom portion of the membrane).

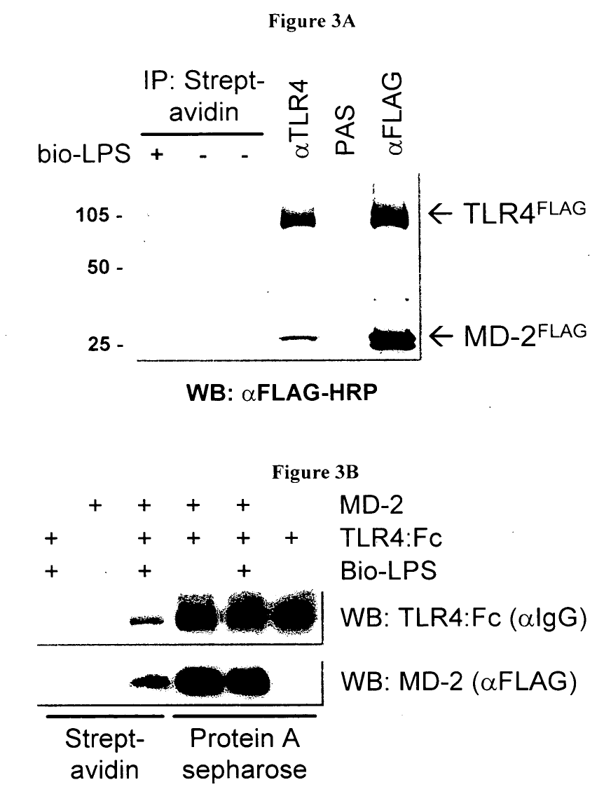

As shown in FIG. 3A (lane 1), biotin-LPS was unable to capture TLR4 in the absence of MD-2. However, the addition of MD-2 enabled LPS recognition, and both molecules were readily detected in the precipitate (FIG. 3A, lane 3). Protein A Sepharose™ beads, which bind to the Fc portion of the IgG₂ₐ molecule, precipitated the chimeric construct and the associated MD-2 (FIG. 3A, lanes 4 and 5). The addition of LPS did not alter the ability of TLR4 to bind MD-2 (FIG. 3A, lane 5).

These results demonstrate that MD-2 and TLR4 can recognize each other without the cooperation of additional proteins of cellular origin; complexes of MD-2 and TLR4 bound to LPS without any other cell associated factors, and LPS did not interfere with the MD-2:TLR4 interaction. Therefore, compounds that interfere with binding of MD-2 to TLR4, but don't affect binding of LPS to MD-2, can still inhibit TLR4 signalling.

### Example 4

### The Ratio of MD-2 to TLR4 on the Cell Surface is 1:1

Since monomeric MD-2 binds to both LPS and TLR4, and the addition of LPS does not change the aggregation status of TLR4 bound MD-2, the question arose as to whether the ratio of TLR4 and MD-2 is unchanged during the LPS recognition event. To do so, cells that stably expressed TLR4^{FLAG} and transiently transfected FLAG tagged MD-2 were used. Since the detection of both TLR4 and MD-2 depended upon the use of the same HRP-conjugated anti-FLAG antibody, the signal intensity of the FLAG positive bands correlated with the amount of each protein present on the blotted membrane.

Protein binding to LPS was studied as described (4). Briefly, the polysaccharide of E. coli's LPS (0111:B4) was labeled using hydrazide-biotin as per the manufacturer instructions (Pierce). Biotinylated LPS was gel filtered in HANK's balanced solution to remove free biotin, tested for activity and stored at 4° C. The assay was designed to detect the interaction of epitope-tagged recombinant proteins with LPS. To detect LPS binding to soluble proteins, biotin-LPS (0.5 or 1 μg/ml) and SAB (20 μl packed resin/point) were mixed with culture supernatants from transfected cells that secrete the candidate proteins for one hour at 37° C. or overnight at 4° C. To detect LPS binding to proteins that are expressed on the surface of cells, biotin-LPS was added to 5 ml of medium covering monolayers of growing cells at a final concentration of 1 μg/ml, and treated for 1 hour in a 37° C. incubator in a 5% CO₂ humidified atmosphere. Cells were lysed as described (4), and lysates were subjected to centrifugation at 10,000×g×5 min to remove insoluble material. LPS-interacting proteins were captured using 20 μl of packed SAB/sample directly added to the post nuclear whole cell lysates. Beads were then extensively washed in lysis buffer and proteins were eluted from the beads by boiling in SDS sample buffer. LPS-precipitated proteins were resolved by SDS-PAGE and Western blotted using antibodies to their epitope tags (e.g., anti-GFP to detect TLR4^{YFP}, anti mouse to detect TLR4:Fc, anti-FLAG to detect MD-2^{FLAG} or TLR4^{FLAG}).

As shown in FIG. 3B, when these cells were exposed to biotin-LPS under conditions that should activate signal transduction, and biotin-LPS was subsequently captured with avidin beads after lysis, two bands of the same intensity were detected in the anti-FLAG western blot (FIG. 3B, lane 1). These results suggest that the ratio of TLR4 and MD-2 in the LPS receptor is 1:1. Briefly, cells expressing both FLAG-tagged TLR4 and FLAG-tagged MD-2 were grown in 10 cm dishes and stimulated with 1 μg of biotin-LPS/ml at 37° C. One hour later, the cells were washed and lysed in detergent. Lysates were incubated with streptavidin beads and the bound proteins were analyzed by western blotting with anti-FLAG mAb.

Identical results were obtained in the soluble phase by using a FLAG tagged extracellular TLR4 chimeric protein (not shown). As a comparison, TLR4 was immunoprecipitated using both the HTA125 (anti-TLR4) or the M2 (anti-FLAG) antibodies. The anti-TLR4 antibody consistently precipitated less MD-2 biotin-LPS (FIG. 3B, lane 4). This result might explain why this monoclonal antibody can inhibit LPS responses (6), i.e., it partially inhibits the association of TLR4 with monomeric MD-2.

These results demonstrate that the ratio of TLR4 and MD-2 in the functional LPS receptor is 1:1.

### Example 5

### LPS does not Alter the Affinity of MD-2 for TLR4

Since MD-2 can be provided to TLR4 as a soluble molecule, the dissociation constant for this interaction was determined.

To determine the K_{d} for the interaction between TLR4 and MD-2, an indirect ELISA-like binding assay was developed. Fifty μl of purified TLR4:Fc at 20 μg/ml (FIG. 4A) or at the concentration indicated in FIG. 4B was plated on protein A coated, high-protein binding 96 well plates. The plates were then washed 3× in PBS-Tween, blocked with 1% BSA, 5% sucrose, 0.1% Tween in PBS for 1 hour, and incubated with baculoviral-derived 6×His tagged MD-2 in PBS at the indicated concentration (FIG. 4A) or at 12 nM (FIG. 4B) in a total volume of 50 μl at 37° C. for one hour. In some experiments, LPS was included at 0.1 μg/ml (FIG. 4A, solid line) or as indicated (FIG. 4B) in the MD-2 titrations. MD-2 bound to TLR4:Fc was detected using Ni-HRP (1:2,000 in PBS-Tween, Sigma), and developed by incubation with its chromogenic substrate per the manufacturer's instructions (DAKO, Carpinteria, Calif.). Each condition was measured in triplicate; the absorbance of each sample was measured at 450 nm; the results are presented as the average reading±SD.

In FIG. 4A, saturating amounts of TLR4:Fc were adsorbed to protein A coated 96 well plates and purified soluble MD-2 was added in titrated amounts. The 6×His tag present at the C terminus of MD-2 was detected using a Ni based HRP labeled reagent, and the presence of MD-2 bound to TLR4 was detected using a chromogenic substrate. A concentration of about 12 nM corresponded to half the saturating concentration of MD-2 (FIG. 4A, solid line). Since MD-2 can bind to soluble LPS, and can act as an activating ligand for TLR4, it was conceivable that ligated MD-2 has an altered affinity for TLR4. An identical titration was therefore performed in the presence of 1 μg of E. coli LPS/ml (FIG. 4A, scattered line).

An alternative approach to examine MD-2 binding to TLR4 further substantiated the impression that MD-2 does not alter the binding of LPS to TLR4. In this experiment, the TLR4:Fc protein was adsorbed on protein A coated plastic in titrated amounts. MD-2 was then added at its Kd concentration (12 nM), where changes in binding avidity could be most accurately measured, in the absence or in the presence of increasing amounts of LPS (0, 0.1, 1 and 10 ng/ml). As shown in FIG. 4B, the addition of LPS to MD-2, did not significantly change the Kd of the binding, suggesting that MD-2 binding to TLR4 is independent from its interactions with LPS.

These results demonstrate that these methods can be used to detect and quantify MD-2 binding to TLR4.

### Example 6

### E5564 Inhibits LPS Binding to TLR4

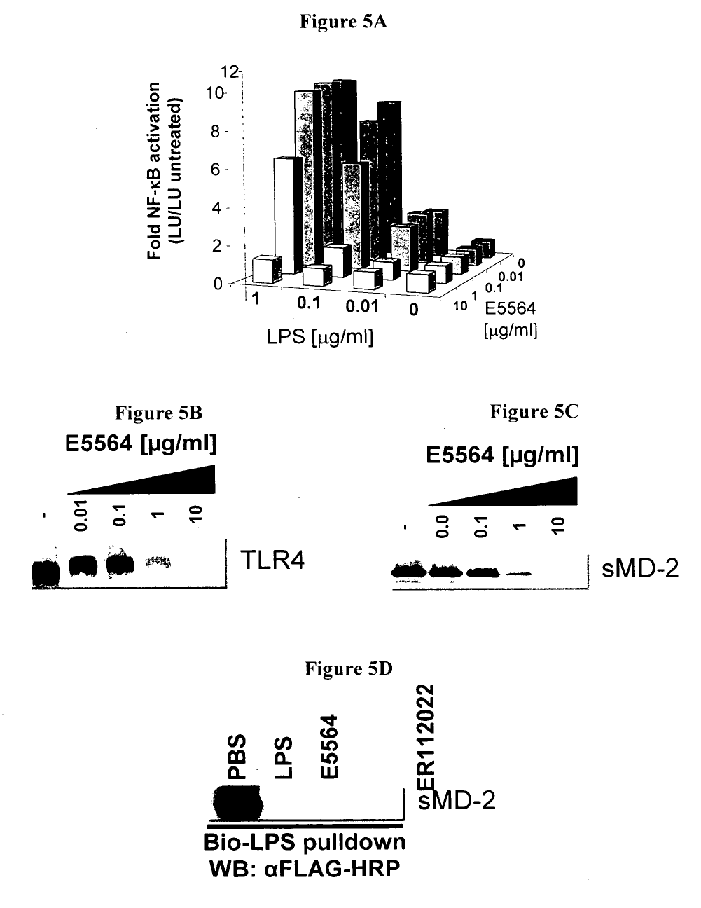

E5564 is a synthetic LPS antagonist similar in structure to R. capsulatum lipid A (24). Saitoh and colleagues have previously reported that E5564 prevents LPS-induced TLR4 dimerization (3). Since it was hypothesized that MD-2 is the LPS-binding portion of the LPS receptor, it was predicted that the inhibitory effects of E5564 on endotoxin-induced stimulation are due to competitive inhibition for a binding site on MD-2. As shown in FIG. 5A, the optimal concentration of E5564 necessary to achieve complete inhibition of LPS-induced responses was first determined. HEK293 cells stably expressing TLR4^{YFP} and MD-2^{FLAG} were transiently transfected with a NF-κB luciferase reporter plasmid and seeded on a 96 well plate at a density of ˜50,000 cells/well. The cells were then stimulated with increasing amounts of LPS (x axis, from right to left) in the presence of increasing amounts of the LPS antagonist E5564 (y axis, dark to light bars). After an overnight incubation, luciferase activity was measured using a multiplate luminometer and shown as in FIG. 1A. E5564 consistently abrogated LPS signaling when used tenfold in excess (weight/vol) (FIG. 5A).

Titrated amounts of E5564 were then tested as competitors for biotin-LPS (0.5 μg/ml) binding to soluble MD-2 or cell associated TLR4. HEK293 cells stably expressing TLR4^{YFP} and MD-2^{FLAG} were plated in 10 cm dishes and treated with biotin-LPS (0.5 μg/ml) for one hour at 37° C. in the absence (FIG. 5B, lane 1) or presence (FIG. 5B, lanes 2-5) of increasing amounts of the LPS antagonist E5564. Biotinylated LPS was then captured in the lysates using streptavidin beads, eluted and resolved in a reducing SDS-PAGE. TLR4^{YFP} was detected by Western blotting with an anti-GFP mAb.

A similar experiment, shown in FIG. 5C, was performed by adding biotin-LPS plus variable amounts of E5564 to conditioned medium containing soluble MD-2 (FIG. 5C, 10 ml/lane); sMD-2 was precipitated with streptavidin beads and analyzed by western blot with an anti-FLAG mAb as in FIG. 3A.

Finally, binding of biotin-LPS to soluble MD-2 (FIG. 5D, lane 1) was evaluated in the presence of varying ratios (w/v) of non labeled LPS (FIG. 5D, lane 2), E5564 (FIG. 5D, lane 3) or the synthetic TLR4 agonist, ER112022 (FIG. 5D, lane 4). Note that the conditioned medium in which these experiments were performed contained 5% fetal calf serum as a source of sCD14 and LBP.

As predicted by the functional titration, E5564 efficiently displaced biotin-LPS binding to soluble MD-2 when present in tenfold (w/v) excess (FIG. 5B). Similarly, when LPS binding to MD-2 was assessed using cells that express TLR4/MD-2, the inhibitor displaced LPS in a dose dependent manner, resulting in the inability to precipitate MD-2 bound TLR4 (FIG. 5C). As expected, unlabeled LPS also inhibited biotin-labeled LPS binding to MD-2. Previous studies established that acyclic analogs of lipid A (i.e., analogs that do not contain the diglucosamine backbone), such as the synthetic compound known as ER112022, are pharmacologically similar to complete LPS. As one might have predicted based on the above experiments, ER112022 could displace biotin-LPS from MD-2 as well (FIG. 5D). Deacylated LPS, in which the lipid A moiety has been subjected to base hydrolysis, is neither a TLR4 agonist nor an LPS antagonist (Seid and Sadoff, 1981, J Biol Chem 256:7305; Von Eschen and Rudbach, 1976, J Immunol 116:8). Deacylated LPS bound to MD-2, but failed to displace fully acylated LPS (not shown).

Hence, the ability of a ligand to bind to MD-2 is a prerequisite for TLR4 activity either stimulatory or inhibitory. The ability of the ligand to subsequently activate a signal transduction program is presumably the result of an alteration in the conformation of MD-2 that results in a change in aggregation status of TLR4, and the recruitment of TIR-domain adapter molecules to the “receptosome” (26).

These results demonstrate that these methods can be used to detect and quantify LPS binding to MD-2, and so are useful in screening for compounds that affect LPS/MD-2 interaction.

### Example 7

### Human Normal Serum Contains Soluble MD-2 at a Concentration of 1.5 nM (45 ng/ml)

MD-2 research has been plagued by the lack of reagents, particularly monoclonal antibodies that could be used to detect native protein. Efforts to establish monoclonal and polyclonal antibodies able to recognize endogenous soluble MD-2 have, to date, only been marginally successful. For example, while antibodies provided by Viriyakosol and colleagues (clone 5H10 (9), and a rabbit polyclonal antiserum) proved efficient in detecting baculoviral MD-2, it was not possible to detect the endogenous protein in serum by immunoprecipitation or Western blot. MD-2 in the lysates of LPS-responsive immune cells or even transfected HEK293 was also undetectable (data not shown). Similarly, other available polyclonal (Imgenex, Santa Cruz) or monoclonal (e-Bioscience) antibodies failed to detect native MD-2. Therefore, an alternative strategy was developed to demonstrate the presence of soluble MD-2 in human serum.

Human serum was collected from healthy volunteers using standard serum Vacutainers™ blood collection tubes (Becton Dickinson) and clotted for 30 minutes at room temperature. The blood clot was removed by centrifugation and serum stored at −20° C. in 1 ml aliquots until use. MD-2 was removed from serum using immunoaffinity techniques using chimeric antibody-like proteins bound to protein A Sepharose™ beads (PAS). Human antibodies can potentially compete with the TLR chimeras for binding sites on protein A beads. Therefore, they were removed from serum by two preclearing steps, of two hours each, with 1/10 the volume of packed PAS beads.

HEK293 cells that had been transduced with retrovirus encoding the TLR:Fc fusion proteins were grown in protein free medium and served as the source of conditioned medium. TLR4:Fc and TLR2:Fc were captured from 50-100 ml of medium using 40 μl of packed PAS/ml for one hour at 4° C. Coated beads were then added to sera that had been precleared with PAS alone for 1 hour at 4° C. Human serum was diluted in DMEM to a final concentration of 20% v/v before use on reporter cells. Before each conjugation/treatment step, the beads were washed with 20% ethanol in PBS followed by equilibration in DMEM. Reconstitution with MD-2 was performed by adding the indicated amounts of recombinant purified MD-2 to the TLR4:Fc depleted stimulating media.

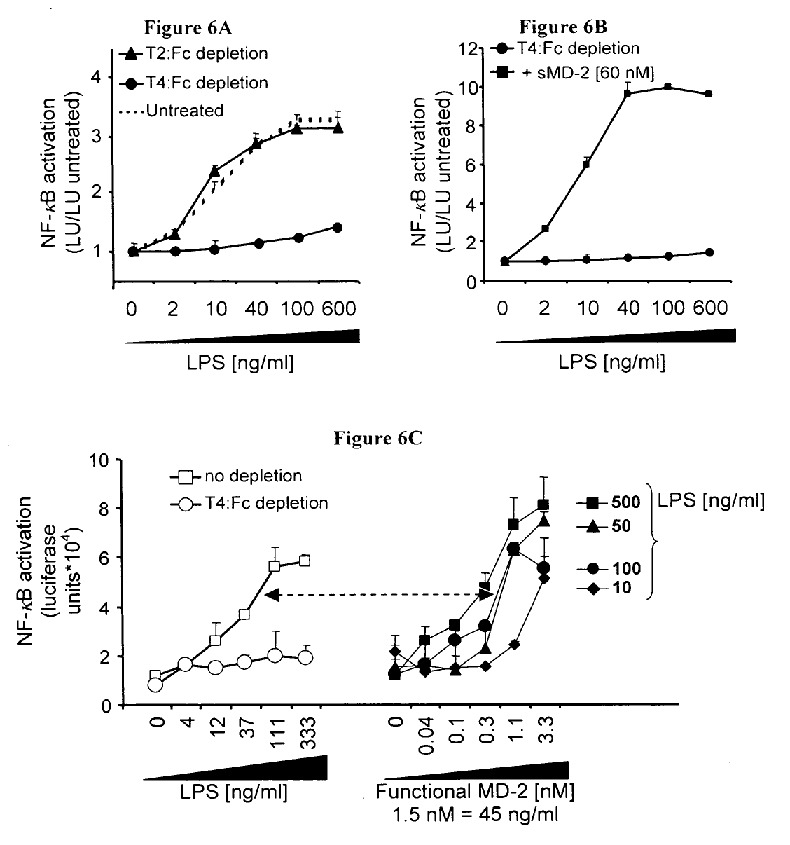

To determine whether TLR4 can efficiently bind to monomeric soluble MD-2 in whole blood or serum, TLR4:Fc chimeric protein immobilized on protein A sepharose beads was used to deplete MD-2 from the serum of healthy human donors from endogenous sMD-2. Depleted sera were then tested for the ability to confer LPS responses in TLR4/KB-Luc 293 reporter cells. Human serum, incubated with protein A beads only and used at a final concentration of 20% in DMEM, enabled LPS responsiveness to TLR4 expressing cells up to 3 to 4 fold when compared to unstimulated cells (FIG. 6A, dashed line; representative of 3 separate donors). Mock depletion of serum MD-2 with TLR2:Fc (FIG. 6A, triangles) did not alter the response. However, pretreatment of the serum with the TLR4:Fc chimera completely abrogated the serum-enhanced response, due to the elimination of soluble endogenous monomeric MD-2 (circles). To confirm that loss of function was attributable to loss of endogenous soluble MD-2, the TLR4:Fc treated serum was reconstituted by adding purified recombinant baculoviral-derived MD-2. Addition of exogenous sMD-2 restored (and even enhanced) LPS responsiveness (squares), suggesting that TLR4:Fc depleted serum of an enhancing capability that was identical in function to sMD-2.

To quantify sMD-2 in human serum, TLR4:Fc treated serum was reconstituted with increasing concentration of MD-2 and sought to determine the amount of purified recombinant MD-2 required to functionally match the physiological activation levels conferred by untreated human serum at the same LPS concentration. TLR4 expressing 293 cells were stimulated in TLR4 pretreated human serum (20% in DMEM) from donor AV in the presence of increasing amounts of MD-2 in four fixed concentrations of LPS (500, 100, 50, 10 ng/ml, left portion of FIG. 6B). The activation corresponding to a concentration of LPS of 50 ng/ml is indicated with the arrow. At this concentration of LPS (between the filled triangles and filled circles), approximately 1.5 nM baculoviral MD-2 (in MD-2 depleted serum) conferred a comparable response. Thus, the concentration of soluble monomeric MD-2 in normal human serum is approximately 1.5 nM, i.e. 45 ng/ml.

These results demonstrate that these methods can be used to detect and quantify MD-2 levels in serum, and that a threshold level of MD-2 in health people is likely to be about 1.5 nM.

### Example 8

### Purified TLR4 Ectodomain Inhibits the Effects of LPS by Neutralizing MD-2

To date, there has been no direct evidence that LPS binds directly to TLR4, although previous molecular genetic studies with pharmacological antagonists of LPS suggested such an interaction (29, 30). Instead, most of the evidence suggests that MD-2 is the binding portion of the TLR4/MD-2 signal transduction complex. Thus, any therapeutic strategy of inhibiting the effects of LPS during clinical disease by infusing large amounts of TLR4 ectodomain, in the hopes of binding and neutralizing LPS, seem unrealistic. On the other hand, if the access of MD-2 to TLR4 were a rate limiting step in the initiation of LPS signaling, it was reasoned that excess amounts of the TLR4 ectodomain would inhibit endotoxin responses by preventing the interaction of MD-2/LPS with surface TLR4.

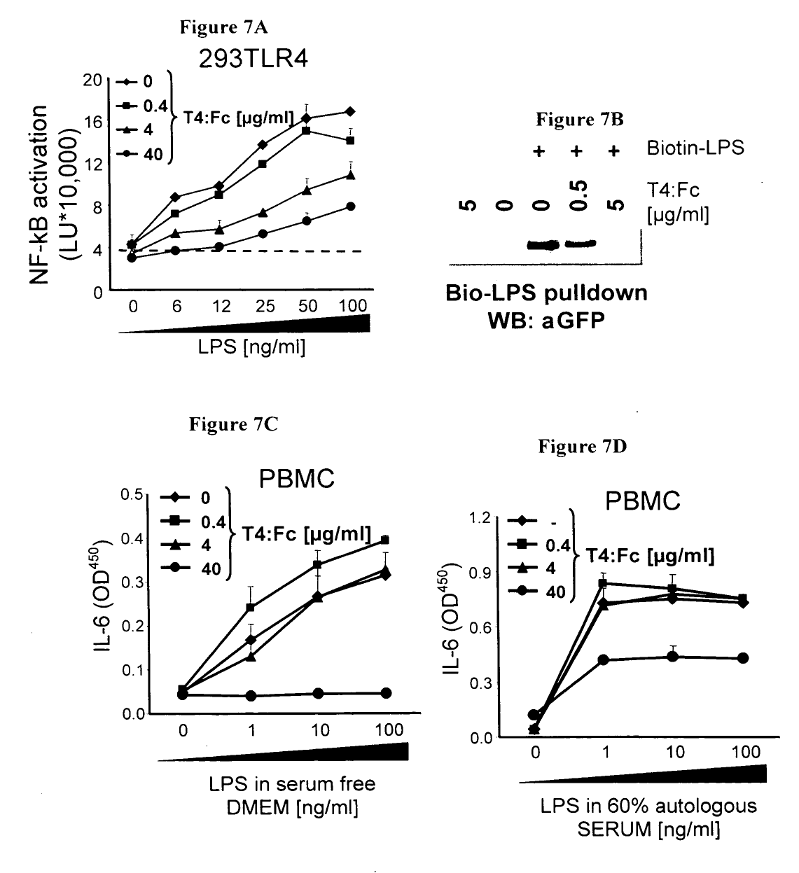

Thus, the TLR4:Fc fusion construct was purified to near homogeneity. This fusion protein, denoted as TLR4:Fc, was tested to see if it could inhibit the effects of LPS in TLR4-transfected HEK293 cells to which recombinant MD-2 was added as a tissue culture supernatant from MD-2 expressing cells. Cells were transfected with an NF-κB reporter construct, and the following day were stimulated with increasing amounts of LPS. As can be seen from FIG. 7A, LPS responses in these cells were inhibited when the concentration of TLR4 was 4 μg/ml and nearly completely inhibited at a concentration only 10-fold higher.

Similarly, the Fc fusion protein was tested with human PBMC under serum free conditions, where the only MD-2 was cell bound on the surface of monocytes. Under these conditions, TLR4:Fc was again capable of inhibiting the effects of LPS, although LPS binding studies to TLR4:Fc under identical serum-free conditions failed to show any direct interaction of the TLR4 ectodomain with endotoxin (FIG. 7C). When 60% autologous serum was added to the PBMC, TLR4 was still capable of inhibiting the effects of LPS, albeit to a somewhat attenuated degree due to the presence of LBP, soluble CD14 and, of course, sMD-2 (FIG. 7D).

To determine if the effects of TLR4:Fc were, in fact, due to its interactions with MD-2 (rather than LPS), and, as a result preventing the formation of a functional LPS receptor, HEK/TLR4^{YFP} cells were plated and cultured overnight. The cells were washed the next day in protein free medium, and fresh supernatants from MD-2 transduced HEK293 cells were added as a source of soluble MD-2 in the absence or presence of TLR4:Fc. Biotinylated LPS was then added, and the binding of LPS to MD-2 was evaluated as a means to precipitate full length YFP-tagged TLR4. These monolayers were washed, lysed and subjected to precipitation with streptavidin beads. The precipitants were then analyzed by immunoblotting against YFP (FIG. 7b). Streptavidin failed to pull down the full length TLR4^{YFP} when TLR4:Fc was present (FIG. 7B, lane 5).

These results indicate that the TLR4:Fc fusion protein inhibited the ability of MD-2 to bind TLR4, and is therefore useful in modulating TLR4 signalling.

### REFERENCES

1. Bell, J. K., G. E. Mullen, C. A. Leifer, A. Mazzoni, D. R. Davies, and D. M. Segal. 2003. Leucine-rich repeats and pathogen recognition in Toll-like receptors. Trends Immunol 24:528.
2. Weber, A. N., S. Tauszig-Delamasure, J. A. Hoffmann, E. Lelievre, H. Gascan, K. P. Ray, M. A. Morse, J. L. Imler, and N. J. Gay. 2003. Binding of the Drosophila cytokine Spatzle to Toll is direct and establishes signaling. Nat Immunol 4:794.
3. Saitoh, S., S. Akashi, T. Yamada, N. Tanimura, M. Kobayashi, K. Konno, F. Matsumoto, K. Fukase, S. Kusumoto, Y. Nagai, Y. Kusumoto, A. Kosugi, and K. Miyake. 2004. Lipid A antagonist, lipid IVa, is distinct from lipid A in interaction with Toll-like receptor 4 (TLR4)-MD-2 and ligand-induced TLR4 oligomerization. Int Immunol 16:961.
4. Visintin, A., E. Latz, B. G. Monks, T. Espevik, and D. T. Golenbock. 2003. Lysines 128 and 132 enable lipopolysaccharide binding to MD-2, leading to Toll-like receptor-4 aggregation and signal transduction. J Biol Chem 278:48313.
5. Beutler, B. 2004. Inferences, questions and possibilities in Toll-like receptor signalling. Nature 430:257.
6. Shimazu, R., S. Akashi, H. Ogata, Y. Nagai, K. Fukudome, K. Miyake, and M. Kimoto. 1999. MD-2, a molecule that confers lipopolysaccharide responsiveness on Toll-like receptor 4. J Exp Med 189:1777.
7. Nagai, Y., S. Akashi, M. Nagafuku, M. Ogata, Y. Iwakura, S. Akira, T. Kitamura, A. Kosugi, M. Kimoto, and K. Miyake. 2002. Essential role of MD-2 in LPS responsiveness and TLR4 distribution. Nat Immunol.
8. Schromm, A. B., E. Lien, P. Henneke, J. C. Chow, A. Yoshimura, H. Heine, E. Latz, B. G. Monks, D. A. Schwartz, K. Miyake, and D. T. Golenbock. 2001. Molecular genetic analysis of an endotoxin nonresponder mutant cell line: a point mutation in a conserved region of MD-2 abolishes endotoxin-induced signaling. J Exp Med 194:79.
9. Viriyakosol, S., P. S. Tobias, R. L. Kitchens, and T. N. Kirkland. 2001. MD-2 Binds to Bacterial Lipopolysaccharide. J Biol Chem 276:38044.
10. Latz, E., A. Visintin, E. Lien, K. A. Fitzgerald, T. Espevik, and D. T. Golenbock. 2003. The LPS receptor generates inflammatory signals from the cell surface. J Endotoxin Res 9:375.
11. Latz, E., A. Visintin, E. Lien, K. A. Fitzgerald, B. G. Monks, E. A. Kurt-Jones, D. T. Golenbock, and T. Espevik. 2002. Lipopolysaccharide rapidly traffics to and from the Golgi apparatus with the toll-like receptor 4-MD-2-CD14 complex in a process that is distinct from the initiation of signal transduction. J Biol Chem 277:47834.
12. Akashi, S., S. Saitoh, Y. Wakabayashi, T. Kikuchi, N. Takamura, Y. Nagai, Y. Kusumoto, K. Fukase, S. Kusumoto, Y. Adachi, A. Kosugi, and K. Miyake. 2003. Lipopolysaccharide interaction with cell surface Toll-like receptor 4-MD-2: higher affinity than that with MD-2 or CD14. J Exp Med 198:1035.
13. Inohara, N., and G. Nunez. 2002. ML—a conserved domain involved in innate immunity and lipid metabolism. Trends Biochem Sci 27:219.
14. Gangloff, M., and N. J. Gay. 2004. MD-2: the Toll ‘gatekeeper’ in endotoxin signalling. Trends Biochem Sci 29:294.
15. Gruber, A., M. Mancek, H. Wagner, C. J. Kirschning, and R. Jerala. 2004. Structural model of MD-2 and functional role of its basic amino acid clusters involved in cellular lipopolysaccharide recognition. J Biol Chem 279:28475.
16. Mullen, G. E., M. N. Kennedy, A. Visintin, A. Mazzoni, C. A. Leifer, D. R. Davies, and D. M. Segal. 2003. The role of disulfide bonds in the assembly and function of MD-2. Proc Natl Acad Sci USA 100:3919.
17. Visintin, A., A. Mazzoni, J. A. Spitzer, and D. M. Segal. 2001. Secreted MD-2 is a large polymeric protein that efficiently confers lipopolysaccharide sensitivity to Toll-like receptor 4. Proc Natl Acad Sci USA 98:12156.
18. Re, F., and J. L. Strominger. 2002. Monomeric recombinant MD-2 binds TLR4 tightly and confers LPS responsiveness. J Biol Chem.
19. Kennedy, M. N., G. E. Mullen, C. A. Leifer, C. Lee, A. Mazzoni, K. N. Dileepan, and D. M. Segal. 2004. A complex of soluble MD-2 and lipopolysaccharide serves as an activating ligand for Toll-like receptor 4. J Biol Chem 279:34698.
20. Jack, R. S., X. Fan, M. Bernheiden, G. Rune, M. Ehlers, A. Weber, G. Kirsch, R. Mentel, B. Furll, M. Freudenberg, G. Schmitz, F. Stelter, and C. Schutt. 1997. Lipopolysaccharide-binding protein is required to combat a murine gram-negative bacterial infection. Nature 389:742.
21. Wurfel, M. M., B. G. Monks, R. R. Ingalls, R. L. Dedrick, R. Delude, D. Zhou, N. Lamping, R. R. Schumann, R. Thieringer, M. J. Fenton, S. D. Wright, and D. Golenbock. 1997. Targeted deletion of the lipopolysaccharide (LPS)-binding protein gene leads to profound suppression of LPS responses ex vivo, whereas in vivo responses remain intact. J Exp Med 186:2051.
22. Perera, P. Y., S. N. Vogel, G. R. Detore, A. Haziot, and S. M. Goyert. 1997. CD14-dependent and CD 14-independent signaling pathways in murine macrophages from normal and CD14 knockout mice stimulated with lipopolysaccharide or taxol. J Immunol 158:4422.
23. Wang, R. F., X. Wang, S. L. Johnston, G. Zeng, P. F. Robbins, and S. A. Rosenberg. 1998. Development of a retrovirus-based complementary DNA expression system for the cloning of tumor antigens. Cancer Res 58:3519.
24. Mullarkey, M., J. R. Rose, J. Bristol, T. Kawata, A. Kimura, S. Kobayashi, M. Przetak, J. Chow, F. Gusovsky, W. J. Christ, and D. P. Rossignol. 2003. Inhibition of endotoxin response by e5564, a novel Toll-like receptor 4-directed endotoxin antagonist. J Pharmacol Exp Ther 304:1093.
25. Fitzgerald, K. A., D. C. Rowe, B. J. Barnes, D. R. Caffrey, A. Visintin, E. Latz, B. Monks, P. M. Pitha, and D. T. Golenbock. 2003. LPS-TLR4 signaling to IRF-3/7 and NF-kappaB involves the toll adapters TRAM and TRIF. J Exp Med 198:1043.
26. Visintin, A., E. Latz, B. G. Monks, T. Espevik, and D. T. Golenbock. 2003. Lysines 128 and 132 enable lipopolysaccharide binding to MD-2, leading to Toll-like receptor-4 aggregation and signal transduction. J Biol Chem 278:48313.
27. Triantafilou, M., K. Brandenburg, S. Kusumoto, K. Fukase, A. Mackie, U. Seydel, and K. Triantafilou. 2004. Combinational clustering of receptors following stimulation by bacterial products determines lipopolysaccharide responses. Biochem J 381:527.
28. Pfeiffer, A., A. Bottcher, E. Orso, M. Kapinsky, P. Nagy, A. Bodnar, I. Spreitzer, G. Liebisch, W. Drobnik, K. Gempel, M. Horn, S. Holmer, T. Hartung, G. Multhoff, G. Schutz, H. Schindler, A. J. Ulmer, H. Heine, F. Stelter, C. Schutt, G. Rothe, J. Szollosi, S. Damjanovich, and G. Schmitz. 2001. Lipopolysaccharide and ceramide docking to CD14 provokes ligand-specific receptor clustering in rafts. Eur J Immunol 31:3153.
29. Lien, E., T. K. Means, H. Heine, A. Yoshimura, S. Kusumoto, K. Fukase, M. J. Fenton, M. Oikawa, N. Qureshi, B. Monks, R. W. Finberg, R. R. Ingalls, and D. T. Golenbock. 2000. Toll-like receptor 4 imparts ligand-specific recognition of bacterial lipopolysaccharide. J. Clin. Invest. 105:497.
30. Poltorak, A., P. Ricciardi-Castagnoli, S. Citterio, and B. Beutler. 2000. Physical contact between lipopolysaccharide and Toll-like receptor 4 revealed by genetic complementation. Proc. Natl. Acad. Sci. U.S.A. 97:2163.
31. Steeghs, L., R. den Hartog, A. den Boer, B. Zomer, P. Roholl, and P. van der Ley. 1998. Meningitis bacterium is viable without endotoxin. Nature 392:449.
32. Somerville, J. E., Jr., L. Cassiano, B. Bainbridge, M. D. Cunningham, and R. P. Darveau. 1996. A novel Escherichia coli lipid A mutant that produces an antiinflammatory lipopolysaccharide. J Clin Invest 97:359.
33. Lien, E., T. K. Means, H. Heine, A. Yoshimura, S. Kusumoto, K. Fukase, M. J. Fenton, M. Oikawa, N. Qureshi, B. Monks, R. W. Finberg, R. R. Ingalls, and D. T. Golenbock. 2000. Toll-like receptor 4 imparts ligand-specific recognition of bacterial lipopolysaccharide. J Clin Invest 105:497.
34. Pugin, J., S. Stern-Voeffray, B. Daubeuf, M. A. Matthay, G. Elson, and I. Dunn-Siegrist. 2004. Soluble MD-2 activity in plasma from patients with severe sepsis and septic shock. Blood 104:4071.
35. Brennan, C. A., and K. V. Anderson. 2004. Drosophila: the genetics of innate immune recognition and response. Annu Rev Immunol 22:457.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating or preventing a disorder associated with a gram negative bacterial infection in a subject, the method comprising administering to the subject a therapeutically effective amount of a composition comprising an extracellular domain of Toll-Like Receptor 4 (TLR4).

2. The method of claim 1, comprising administering a fusion protein comprising an extracellular domain of TLR4 fused to another protein.

3. The method of claim 2, wherein the other protein is an IgG Fc fragment.

4. The method of claim 1, wherein the subject is at risk for developing sepsis.

5. The method of claim 4, wherein the subject has penetrating trauma to the abdomen, heart valve disease, or a large bowel incarceration.

6. The method of claim 1, wherein the subject has one or more symptoms of sepsis.

7. The method of claim 6, wherein the symptom of sepsis is selected from the group consisting of shaking, chills, fever, weakness, confusion, nausea, vomiting, and diarrhea.

8. The method of claim 1, wherein the subject has one or more symptoms of septic shock.

9. The method of claim 8, wherein the symptom of septic shock is selected from the group consisting of confusion and decreased consciousness; shaking chills; a rapid rise in or lower than normal temperature; warm, flushed skin; a rapid, pounding pulse; excessively rapid breathing; blood pressure that rises and falls; and extremities that are cool, pale, and bluish.

10. A method of removing soluble Myeloid Differentiation Antigen-2 (sMD-2) from the blood of a subject, the method comprising:
removing blood from the subject;
contacting the blood with a composition comprising an extracellular domain of Toll-Like Receptor 4 (TLR4) under conditions and for a time sufficient to bind sMD-2 in the blood to the TLR4, thereby forming TLR4/MD-2 complexes;
removing the TLR4/MD-2 complexes from the blood; and
optionally returning the blood to the subject,
thereby removing soluble MD-2 from the blood of the subject.

11. The method of claim 10, wherein the TLR4 is bound to a collectible substrate.

12. The method of claim 11, wherein the collectible substrate is a bead.

13. The method of claim 10, wherein the TLR4 is bound to a column.

14. The method of claim 10, wherein the composition comprises a TLR4:Fc fusion protein.

15. A method of diagnosing a subject with a gram negative bacterial infection, the method comprising measuring levels of soluble Myeloid Differentiation Antigen-2 (sMD-2) in a sample from the subject, wherein an elevated level of sMD-2 as compared to a reference indicates that the subject has a gram negative bacterial infection.

16. The method of claim 15, wherein the sample comprises a biological fluid.

17. The method of claim 15, wherein the reference is at least about 1.5 nM soluble MD-2 (sMD-2).
